(19) United States
(12) Patent Application Publication
(10) Pub. No.: US 2008/0045507 A1
(43) Pub. Date: Feb. 21, 2008

(54) SUBSTITUTED BENZAMIDE MODULATORS OF THE HISTAMINE H3 RECEPTOR

(76) Inventors: Brett D. Allison, Del Mar, CA (US); Nicholas I. Carruthers, Poway, CA (US); Michael A. Letavic, San Diego, CA (US); Alejandro Santillan, San Diego, CA (US); Chandravadan R. Shah, San Diego, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/766,144

(22) Filed: Jun. 21, 2007

Related U.S. Application Data
(60) Provisional application No. 60/806,164, filed on Jun. 29, 2006.

(51) Int. Cl. A61K 31/495; A61K 31/551; A61P 25/00; A61P 25/06; A61P 25/16; A61P 25/18; A61P 25/24; A61P 25/28; A61P 27/00; A61P 3/04; A61P 31/00; C07D 243/08; C07D 295/14

(52) U.S. Cl. 514/218; 514/255.01; 540/575; 544/391

(57) ABSTRACT

Certain substituted benzamide compounds are histamine H₃ receptor modulators useful in the treatment of histamine H₃ receptor-mediated diseases.

### CROSS-REFERENCE TO RELATED APPLICATIONS

This application claims the benefit of U.S. Provisional Application 60/806,164, filed Jun. 29, 2006, which is hereby incorporated by reference in its entirety.

### FIELD OF THE INVENTION

The present invention relates to certain substituted benzamide compounds, pharmaceutical compositions containing them, and methods of using them for the treatment of disease states, disorders, and conditions mediated by the histamine H₃ receptor.

### BACKGROUND OF THE INVENTION

The histamine H₃ receptor was first described as a presynaptic autoreceptor in the central nervous system (CNS) (Arrang, J.-M. et al., Nature 1983, 302, 832-837) controlling the synthesis and release of histamine. The histamine H₃ receptor is primarily expressed in the mammalian central nervous system (CNS), with some minimal expression in peripheral tissues such as vascular smooth muscle.

Thus, several indications for histamine H₃ antagonists and inverse agonists have been proposed based on animal pharmacology and other experiments with known histamine H₃ antagonists (e.g. thioperamide). (See: “The Histamine H₃ Receptor-A Target for New Drugs”, Leurs, R. and Timmerman, H., (Eds.), Elsevier, 1998; Morisset, S. et al., Nature 2000, 408, 860-864.) These include conditions such as cognitive disorders, sleep disorders, psychiatric disorders, and other disorders.

For example, histamine H₃ antagonists have been shown to have pharmacological activity relevant to several key symptoms of depression, including sleep disorders (e.g. sleep disturbances, fatigue, and lethargy) and cognitive difficulties (e.g. memory and concentration impairment), as described above.

Substituted diazepanyl benzamides were described as histamine H₃ receptor antagonists in Intl. Patent Appl. Publ. WO05/040144 (May 6, 2005). Substituted piperazines and diazepanes were described as histamine H₃ receptor modulators in Intl. Patent Appl. Publ. WO03/004480 (Jan. 16, 2003). However, there remains a need for potent histamine H₃ receptor modulators with desirable pharmaceutical properties.

### SUMMARY OF THE INVENTION

Certain substituted benzamide derivatives have now been found to have histamine H₃ receptor modulating activity. Thus, the invention is directed to the general and preferred embodiments defined, respectively, by the independent and dependent claims appended hereto, which are incorporated by reference herein.

In one general aspect the invention relates to a compound of the following Formula (I):

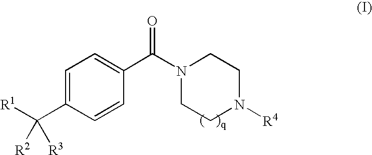

wherein

### R¹ is H, C₁₋₄alkyl, monocyclic C₃₋₇cycloalkyl, or phenyl;

### R² is H or methyl;

or R¹ and R² taken together form monocyclic C₃₋₇cycloalkyl;

### R³ is H, OH, or methyl;

or, when R¹ is not H or phenyl, R² and R³ taken together form a carbonyl;
q is 1 or 2; and

### R⁴ is —C₂₋₆alkyl, —C₃₋₆alkenyl, —C₃₋₆alkynyl, monocyclic cycloalkyl, or —C₁₋₂alkyl-(monocyclic cycloalkyl), each unsubstituted or substituted with —OH, —OC₁₋₄alkyl, fluoro, —NH₂, —NH(C₁₋₄alkyl), or —N(C₁₋₄alkyl)₂;

provided that when R¹ is phenyl, and R² and R³ are both H, then q is 1;
or a pharmaceutically acceptable salt, a pharmaceutically acceptable prodrug,
or a pharmaceutically active metabolite thereof.

In a further general aspect, the invention relates to pharmaceutical compositions each comprising: (a) an effective amount of a compound of Formula (I), or a pharmaceutically acceptable salt, pharmaceutically acceptable prodrug, or pharmaceutically active metabolite thereof; and (b) a pharmaceutically acceptable excipient.

In another general aspect, the invention is directed to a method of treating a subject suffering from or diagnosed with a disease, disorder, or medical condition mediated by histamine H₃ receptor activity, comprising administering to the subject in need of such treatment an effective amount of a compound of Formula (I), or a pharmaceutically acceptable salt, pharmaceutically acceptable prodrug, or pharmaceutically active metabolite thereof.

In certain preferred embodiments of the inventive method, the disease, disorder, or medical condition is selected from: cognitive disorders, sleep disorders, psychiatric disorders, and other disorders.

Additional embodiments, features, and advantages of the invention will be apparent from the following detailed description and through practice of the invention.

### DETAILED DESCRIPTION

The invention may be more fully appreciated by reference to the following description, including the following glossary of terms and the concluding examples. For the sake of brevity, the disclosures of the publications, including patents, cited in this specification are herein incorporated by reference.

As used herein, the terms “including”, “containing” and “comprising” are used herein in their open, non-limiting sense.

The term “alkyl” refers to a straight- or branched-chain alkyl group having from 1 to 12 carbon atoms in the chain. Examples of alkyl groups include methyl (Me, which also may be structurally depicted by /), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples.

The term “cycloalkyl” refers to a saturated or partially saturated, monocyclic, fused polycyclic, or spiro polycyclic carbocycle having from 3 to 12 ring atoms per carbocycle. Illustrative examples of cycloalkyl groups include the following entities, in the form of properly bonded moieties:

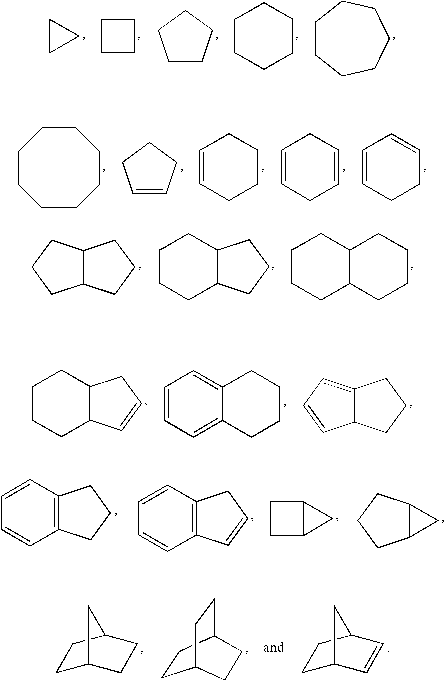

A “heterocycloalkyl” refers to a monocyclic, or fused, bridged, or spiro polycyclic ring structure that is saturated or partially saturated and has from 3 to 12 ring atoms per ring structure selected from carbon atoms and up to three heteroatoms selected from nitrogen, oxygen, and sulfur. The ring structure may optionally contain up to two oxo groups on carbon or sulfur ring members. Illustrative entities, in the form of properly bonded moieties, include:

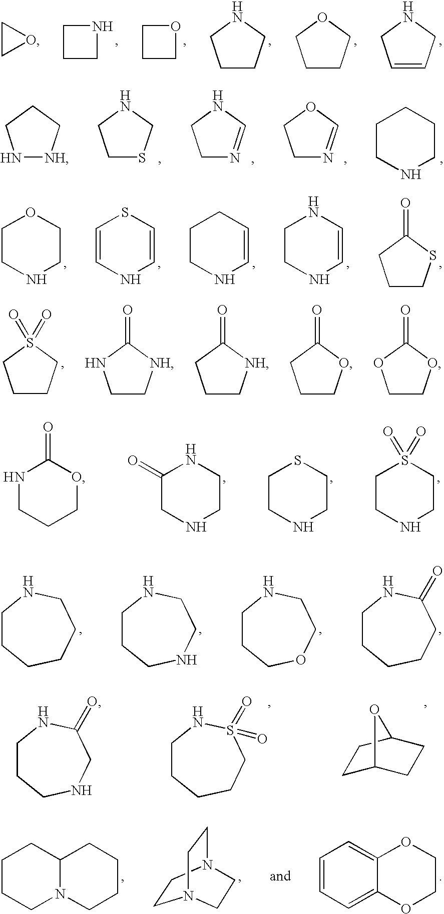

The term “heteroaryl” refers to a monocyclic, fused bicyclic, or fused polycyclic aromatic heterocycle (ring structure having ring atoms selected from carbon atoms and up to four heteroatoms selected from nitrogen, oxygen, and sulfur) having from 3 to 12 ring atoms per heterocycle. Illustrative examples of heteroaryl groups include the following entities, in the form of properly bonded moieties:

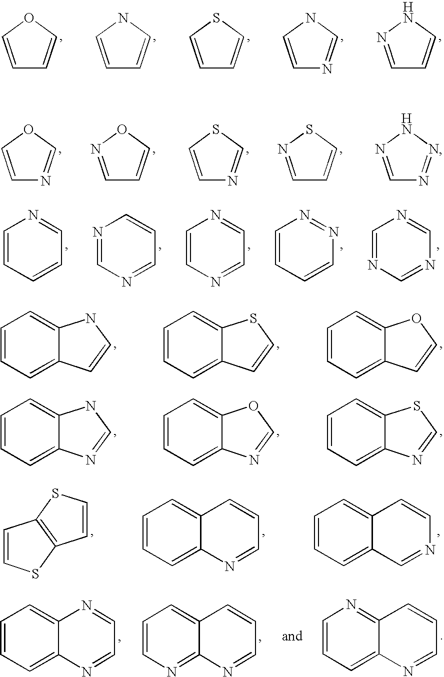

Those skilled in the art will recognize that the species of heteroaryl, cycloalkyl, and heterocycloalkyl groups listed or illustrated above are not exhaustive, and that additional species within the scope of these defined terms may also be selected.

The term “halogen” represents chlorine, fluorine, bromine or iodine. The term “halo” represents chloro, fluoro, bromo or iodo.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system. In cases where a specified moiety or group is not expressly noted as being optionally substituted or substituted with any specified substituent, it is understood that such a moiety or group is intended to be unsubstituted.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers. Additionally, any formula given herein is intended to embrace hydrates, solvates, and polymorphs of such compounds, and mixtures thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, ¹²⁵I, respectively. Such isotopically labeled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the moiety for the variable appearing elsewhere. In other words, where a variable appears more than once, the choice of the species from a specified list is independent of the choice of the species for the same variable elsewhere in the formula.

In preferred embodiments of Formula (I), R¹ is H, methyl, ethyl, propyl, isopropyl, butyl, cyclohexyl, or phenyl.

In preferred embodiments, R² is H.

In preferred embodiments, R¹ and R² taken together form cyclohexyl.

In preferred embodiments, R³ is OH.

In preferred embodiments, R⁴ is ethyl, propyl, isopropyl, sec-butyl, cyclopropyl, cyclobutyl, cyclopentyl, cyclohexyl, cyclopropylmethyl, cyclobutylmethyl, or cyclopentylmethyl, each unsubstituted or substituted as previously described. In further preferred embodiments, R⁴ is isopropyl, cyclopropyl, or cyclobutyl.

In further preferred embodiments, R¹ is H or C₁₋₆alkyl, R² is H, R³ is H or methyl, and R⁴ is cyclopropyl or cyclobutyl.

In certain preferred embodiments, the compound of Formula (I) is selected from the group consisting of:

| Ex. | Chemical Name |
|---|---|
| 1 | [4-(Cyclohexyl-hydroxy-methyl)-phenyl]-(4-isopropyl-piperazin- |
| | 1-yl)-methanone; |
| 2 | [4-(1-Hydroxy-propyl)-phenyl]-(4-isopropyl-piperazin-1-yl)- |
| | methanone; |
| 3 | [4-(Hydroxy-phenyl-methyl)-phenyl]-(4-isopropyl-piperazin-1-yl)- |
| | methanone; |
| 4 | [4-(1-Hydroxy-ethyl)-phenyl]-(4-isopropyl-piperazin-1-yl)- |
| | methanone; |
| 5 | [4-(1-Hydroxy-2-methyl-propyl)-phenyl]-(4-isopropyl-piperazin- |
| | 1-yl)-methanone; |
| 6 | (4-Hydroxymethyl-phenyl)-(4-isopropyl-piperazin-1-yl)-methanone; |
| 7 | [4-(Cyclohexyl-hydroxy-methyl)-phenyl]-(4-isopropyl- |
| | [1,4]diazepan-1-yl)-methanone; |
| 8 | (4-Hydroxymethyl-phenyl)-(4-isopropyl-[1,4]diazepan-1-yl)- |
| | methanone; |
| 9 | (4-Cyclohexanecarbonyl-phenyl)-(4-isopropyl-[1,4]diazepan-1-yl)- |
| | methanone; |
| 10 | [4-(1-Hydroxy-propyl)-phenyl]-(4-isopropyl-[1,4]diazepan-1-yl)- |
| | methanone; |
| 11 | [4-(Hydroxy-phenyl-methyl)-phenyl]-(4-isopropyl-[1,4]diazepan- |
| | 1-yl)-methanone; |
| 12 | [4-(1-Hydroxy-ethyl)-phenyl]-(4-isopropyl-[1,4]diazepan-1-yl)- |
| | methanone; |
| 13 | [4-(1-Hydroxy-2-methyl-propyl)-phenyl]-(4-isopropyl-[1,4]diazepan- |
| | 1-yl)-methanone; |
| 14 | (4-Cyclobutyl-piperazin-1-yl)-[4-(hydroxy-phenyl-methyl)-phenyl]- |
| | methanone; |
| 15 | (4-Cyclobutyl-piperazin-1-yl)-[4-(1-hydroxy-propyl)-phenyl]- |
| | methanone; |
| 16 | (4-Cyclobutyl-piperazin-1-yl)-[4-(1-hydroxy-2-methyl-propyl)- |
| | phenyl]-methanone; |
| 17 | (4-Cyclobutyl-piperazin-1-yl)-[4-(cyclohexyl-hydroxy-methyl)- |
| | phenyl]-methanone; |
| 18 | (4-Cyclobutyl-piperazin-1-yl)-(4-hydroxymethyl-phenyl)-methanone; |
| 19 | (4-Cyclobutyl-[1,4]diazepan-1-yl)-[4-(1-hydroxy-propyl)-phenyl]- |
| | methanone; |
| 20 | (4-Cyclobutyl-[1,4]diazepan-1-yl)-[4-(cyclohexyl-hydroxy-methyl)- |
| | phenyl]-methanone; |
| 21 | (4-Cyclobutyl-[1,4]diazepan-1-yl)-[4-(hydroxy-phenyl-methyl)- |
| | phenyl]-methanone; |
| 22 | (4-Cyclobutyl-[1,4]diazepan-1-yl)-[4-(1-hydroxy-2-methyl-propyl)- |
| | phenyl]-methanone; |
| 23 | (4-Cyclobutyl-[1,4]diazepan-1-yl)-(4-hydroxymethyl-phenyl)- |
| | methanone; |
| 24 | [4-(Cyclohexyl-hydroxy-methyl)-phenyl]-(4-cyclopropyl-piperazin-1- |
| | yl)-methanone; |
| 25 | (4-Cyclopropyl-piperazin-1-yl)-[4-(hydroxy-phenyl-methyl)-phenyl]- |
| | methanone; |
| 26 | (4-Cyclopropyl-piperazin-1-yl)-[4-(1-hydroxy-2-methyl-propyl)- |
| | phenyl]-methanone; |
| 27 | [4-(Cyclohexyl-hydroxy-methyl)-phenyl]-(4-cyclopropyl- |
| | [1,4]diazepan-1-yl)-methanone; |
| 28 | (4-Cyclopropyl-[1,4]diazepan-1-yl)-(4-hydroxymethyl-phenyl)- |
| | methanone; |
| 29 | (4-Cyclohexanecarbonyl-phenyl)-(4-cyclopropyl-[1,4]diazepan-1-yl)- |
| | methanone; |
| 30 | (4-Cyclopropyl-[1,4]diazepan-1-yl)-[4-(hydroxy-phenyl-methyl)- |
| | phenyl]-methanone; |
| 31 | (4-Cyclopropyl-[1,4]diazepan-1-yl)-[4-(1-hydroxy-propyl)-phenyl]- |
| | methanone; |
| 32 | (4-Cyclopropyl-[1,4]diazepan-1-yl)-[4-(1-hydroxy-2-methyl-propyl)- |
| | phenyl]-methanone; |
| 33 | (4-tert-Butyl-phenyl)-(4-cyclobutyl-piperazin-1-yl)-methanone; |
| 34 | (4-Cyclobutyl-piperazin-1-yl)-(4-ethyl-phenyl)-methanone; |
| 35 | (4-Cyclobutyl-piperazin-1-yl)-(4-isopropyl-phenyl)-methanone; |
| 36 | (4-Cyclobutyl-piperazin-1-yl)-(4-cyclohexyl-phenyl)-methanone; |
| 37 | (4-Benzyl-phenyl)-(4-cyclobutyl-piperazin-1-yl)-methanone; |
| 38 | (4-Cyclobutyl-piperazin-1-yl)-(4-propyl-phenyl)-methanone; |
| 39 | (4-Butyl-phenyl)-(4-cyclobutyl-piperazin-1-yl)-methanone; |
| 40 | (4-Cyclobutyl-piperazin-1-yl)-(4-pentyl-phenyl)-methanone; |
| 41 | (4-Cyclobutyl-piperazin-1-yl)-[4-(1-hydroxy-1-methyl-ethyl)-phenyl]- |
| | methanone; |
| 42 | (4-Cyclobutyl-piperazin-1-yl)-[4-(1-hydroxy-cyclohexyl)-phenyl]- |
| | methanone; |
| 43 | (4-Cyclopropyl-[1,4]diazepan-1-yl)-[4-(1-hydroxy-cyclohexyl)- |
| | phenyl]-methanone; |
| 44 | (4-Cyclopropyl-[1,4]diazepan-1-yl)-[4-(1-hydroxy-cyclopentyl)- |
| | phenyl]-methanone; |
| 45 | (4-Cyclobutyl-piperazin-1-yl)-[4-(1-hydroxy-cyclopentyl)-phenyl]- |
| | methanone; |
| 46 | (4-Cyclopropyl-[1,4]diazepan-1-yl)-[4-(1-hydroxy-cycloheptyl)- |
| | phenyl]-methanone; |
| 47 | [4-(1-Hydroxy-cycloheptyl)-phenyl]-(4-isopropyl-piperazin-1-yl)- |
| | methanone; |
| 48 | (4-Cyclopropyl-piperazin-1-yl)-[4-(1-hydroxy-propyl)-phenyl]- |
| | methanone; |
| 49 | (4-Cyclopropyl-piperazin-1-yl)-(4-hydroxymethyl-phenyl)- |
| | methanone; |
| 50 | (4-Butyl-piperazin-1-yl)-(4-hydroxymethyl-phenyl)-methanone; and |
| 51 | (4-sec-Butyl-piperazin-1-yl)-(4-hydroxymethyl-phenyl)-methanone; |

and pharmaceutically acceptable salts thereof.

The invention includes also pharmaceutically acceptable salts of the compounds of Formula (I), preferably of those described above and of the specific compounds exemplified herein, and methods of treatment using such salts.

A “pharmaceutically acceptable salt” is intended to mean a salt of a free acid or base of a compound represented by Formula (I) that is non-toxic, biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Examples of pharmaceutically acceptable salts are those that are pharmacologically effective and suitable for contact with the tissues of patients without undue toxicity, irritation, or allergic response. A compound of Formula (I) may possess a sufficiently acidic group, a sufficiently basic group, or both types of functional groups, and accordingly react with a number of inorganic or organic bases, and inorganic and organic acids, to form a pharmaceutically acceptable salt. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the compound of Formula (I) contains a basic nitrogen, the desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lacetic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents or acceptable substitutes in light of the ordinary level of skill in this technology.

If the compound of Formula (I) is an acid, such as a carboxylic acid or sulfonic acid, the desired pharmaceutically acceptable salt may be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine, and piperazine, and inorganic salts derived from sodium, calcium, potassium, magnesium, manganese, iron, copper, zinc, aluminum, and lithium.

The invention also relates to pharmaceutically acceptable prodrugs of the compounds of Formula (I), and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I)). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

Examples of prodrugs include compounds having an amino acid residue, or a polypeptide chain of two or more (e.g., two, three or four) amino acid residues, covalently joined through an amide or ester bond to a free amino, hydroxy, or carboxylic acid group of a compound of Formula (I). Examples of amino acid residues include the twenty naturally occurring amino acids, commonly designated by three letter symbols, as well as 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone.

Additional types of prodrugs may be produced, for instance, by derivatizing free carboxyl groups of structures of Formula (I) as amides or alkyl esters. Examples of amides include those derived from ammonia, primary C₁₋₆alkyl amines and secondary di(C₁₋₆alkyl) amines. Secondary amines include 5- or 6-membered heterocycloalkyl or heteroaryl ring moieties. Examples of amides include those that are derived from ammonia, C₁₋₃alkyl primary amines, and di(C₁₋₂alkyl)amines. Examples of esters of the invention include C₁₋₇alkyl, C₅₋₇cycloalkyl, phenyl, and phenyl(C₁₋₆alkyl) esters. Preferred esters include methyl esters. Prodrugs may also be prepared by derivatizing free hydroxy groups using groups including hemisuccinates, phosphate esters, dimethylaminoacetates, and phosphoryloxymethyloxycarbonyls, following procedures such as those outlined in Adv. Drug Delivery Rev. 1996, 19, 115. Carbamate derivatives of hydroxy and amino groups may also yield prodrugs. Carbonate derivatives, sulfonate esters, and sulfate esters of hydroxy groups may also provide prodrugs. Derivatization of hydroxy groups as (acyloxy)methyl and (acyloxy)ethyl ethers, wherein the acyl group may be an alkyl ester, optionally substituted with one or more ether, amine, or carboxylic acid functionalities, or where the acyl group is an amino acid ester as described above, is also useful to yield prodrugs. Prodrugs of this type may be prepared as described in J. Med. Chem. 1996, 39, 10. Free amines can also be derivatized as amides, sulfonamides or phosphonamides. All of these prodrug moieties may incorporate groups including ether, amine, and carboxylic acid functionalities.

The present invention also relates to pharmaceutically active metabolites of the compounds of Formula (I), which may also be used in the methods of the invention. A “pharmaceutically active metabolite” means a pharmacologically active product of metabolism in the body of a compound of Formula (I) or salt thereof. Prodrugs and active metabolites of a compound may be determined using routine techniques known or available in the art. See, e.g., Bertolini, et al., J. Med. Chem. 1997, 40, 2011-2016; Shan, et al., J. Pharm. Sci. 1997, 86 (7), 765-767; Bagshawe, Drug Dev. Res. 1995, 34, 220-230; Bodor, Adv. Drug Res. 1984, 13, 224-331; Bundgaard, Design of Prodrugs (Elsevier Press, 1985); and Larsen, Design and Application of Prodrugs, Drug Design and Development (Krogsgaard-Larsen, et al., eds., Harwood Academic Publishers, 1991).

The compounds of Formula (I) and their pharmaceutically acceptable salts, pharmaceutically acceptable prodrugs, and pharmaceutically active metabolites of the present invention are useful as modulators of the histamine H₃ receptor in the methods of the invention. Accordingly, the invention relates to methods of using the compounds of the invention to treat subjects diagnosed with or suffering from a disease, disorder, or condition mediated by the histamine H₃ receptor, such as those described herein.

The term “treat” or “treating” as used herein is intended to refer to administration of a compound or composition of the invention to a subject for the purpose of effecting a therapeutic or prophylactic benefit through modulation of histamine H₃ receptor activity. Treating includes reversing, ameliorating, alleviating, inhibiting the progress of, lessening the severity of, or preventing a disease, disorder, or condition, or one or more symptoms of such disease, disorder or condition mediated through modulation of histamine H₃ receptor activity. The term “subject” refers to a mammalian patient in need of such treatment, such as a human. “Modulators” include both inhibitors and activators, where “inhibitors” refer to compounds that decrease, prevent, inactivate, desensitize or down-regulate histamine H₃ receptor expression or activity, and “activators” are compounds that increase, activate, facilitate, sensitize, or up-regulate histamine H₃ receptor expression or activity.

Accordingly, the invention relates to methods of using the compounds described herein to treat subjects diagnosed with or suffering from a disease, disorder, or condition mediated by histamine H₃ receptor activity, such as: cognitive disorders, sleep disorders, psychiatric disorders, and other disorders. Symptoms or disease states are intended to be included within the scope of “medical conditions, disorders, or diseases.”

Cognitive disorders include, for example, dementia, Alzheimer's disease (Panula, P. et al., Soc. Neurosci. Abstr. 1995, 21, 1977), cognitive dysfunction, mild cognitive impairment (pre-dementia), attention deficit hyperactivity disorders (ADHD), attention-deficit disorders, and learning and memory disorders (Barnes, J. C. et al., Soc. Neurosci. Abstr. 1993, 19, 1813). Learning and memory disorders include, for example, learning impairment, memory impairment, age-related cognitive decline, and memory loss. H₃ antagonists have been shown to improve memory in a variety of memory tests, including the elevated plus maze in mice (Miyazaki, S. et al. Life Sci. 1995, 57(23), 2137-2144), a two-trial place recognition task (Orsetti, M. et al. Behav. Brain Res. 2001, 124(2), 235-242), the passive avoidance test in mice (Miyazaki, S. et al. Meth. Find. Exp. Clin. Pharmacol. 1995, 17(10), 653-658) and the radial maze in rats (Chen, Z. Acta Pharmacol. Sin. 2000, 21(10), 905-910). Also, in the spontaneously hypertensive rat, an animal model for the learning impairments in attention-deficit disorders, H₃ antagonists were shown to improve memory (Fox, G. B. et al. Behav. Brain Res. 2002, 131(1-2), 151-161).

Sleep disorders include, for example, insomnia, disturbed sleep, narcolepsy (with or without associated cataplexy), cataplexy, disorders of sleep/wake homeostasis, idiopathic somnolence, excessive daytime sleepiness (EDS), circadian rhythm disorders, fatigue, lethargy, jet lag, and REM-behavioral disorder. Fatigue and/or sleep impairment may be caused by or associated with various sources, such as, for example, sleep apnea, perimenopausal hormonal shifts, Parkinson's disease, multiple sclerosis (MS), depression, chemotherapy, or shift work schedules.

Psychiatric disorders include, for example, schizophrenia (Schlicker, E. and Marr, I., Naunyn-Schmiedeberg's Arch. Pharmacol. 1996, 353, 290-294), bipolar disorders, manic disorders, depression (Lamberti, C. et al. Br. J. Pharmacol. 1998, 123(7), 1331-1336; Perez-Garcia, C. et al. Psychopharmacology 1999, 142(2), 215-220) (Also see: Stark, H. et al., Drugs Future 1996, 21(5), 507-520; and Leurs, R. et al., Prog. Drug Res. 1995, 45, 107-165 and references cited therein.), obsessive-compulsive disorder, and post-traumatic stress disorder.

Other disorders include, for example, motion sickness, vertigo (e.g. vertigo or benign postural vertigo), tinitus, epilepsy (Yokoyama, H. et al., Eur. J. Pharmacol. 1993, 234, 129-133), migraine, neurogenic inflammation, eating disorders (Machidori, H. et al., Brain Res. 1992, 590, 180-186), obesity, substance abuse disorders, movement disorders (e.g. restless leg syndrome), and eye-related disorders (e.g. macular degeneration and retinitis pigmentosis).

Particularly, as modulators of the histamine H₃ receptor, the compounds of the present invention are useful in the treatment or prevention of depression, disturbed sleep, narcolepsy, fatigue, lethargy, cognitive impairment, memory impairment, memory loss, learning impairment, attention-deficit disorders, and eating disorders.

In a treatment method according to the invention, an effective amount of a compound according to the invention is administered to a subject suffering from or diagnosed as having such a disease, disorder, or condition. An “effective amount” means an amount or dose sufficient to generally bring about the desired therapeutic or prophylactic benefit in patients in need of such treatment.

Effective amounts or doses of the compounds of the present invention may be ascertained by routine methods such as modeling, dose escalation studies or clinical trials, and by taking into consideration routine factors, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician. An exemplary dose is in the range of from about 0.001 to about 200 mg of compound per kg of subject's body weight per day, preferably about 0.05 to 100 mg/kg/day, or about 1 to 35 mg/kg/day, or about 0.1 to 10 mg/kg daily in single or divided dosage units (e.g., BID, TID, QID). For a 70-kg human, an illustrative range for a suitable dosage amount is from about 0.05 to about 7 g/day, or about 0.2 to about 2.5 g/day.

Once improvement of the patient's disease, disorder, or condition has occurred, the dose may be adjusted for preventative or maintenance treatment.

For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms.

In addition, the compounds of the invention may be used in combination with additional active ingredients in the treatment of the above conditions. In an exemplary embodiment, additional active ingredients are those that are known or discovered to be effective in the treatment of conditions, disorders, or diseases mediated by histamine H₃ receptor activity or that are active against another target associated with the particular condition, disorder, or disease, such as H₁ receptor antagonists, H₂ receptor antagonists, H₃ receptor antagonists, topiramate (Topamax™), and neurotransmitter modulators such as serotonin-norepinephrine reuptake inhibitors, selective serotonin reuptake inhibitors (SSRIs), noradrenergic reuptake inhibitors, non-selective serotonin re-uptake inhibitors (NSSRIs), acetylcholinesterase inhibitors (such as tetrahydroaminoacridine, Donepezil (Aricept™), Rivastigmine, or Galantamine (Reminyl™)), or modafinil. The combination may serve to increase efficacy (e.g., by including in the combination a compound potentiating the potency or effectiveness of a compound according to the invention), decrease one or more side effects, or decrease the required dose of the compound according to the invention.

More particularly, compounds of the invention in combination with modafinil are useful for the treatment of narcolepsy, excessive daytime sleepiness (EDS), Alzheimer's disease, depression, attention-deficit disorders, MS-related fatigue, post-anesthesia grogginess, cognitive impairment, schizophrenia, spasticity associated with cerebral palsy, age-related memory decline, idiopathic somnolence, or jet-lag. Preferably, the combination method employs doses of modafinil in the range of about 20 to 300 mg per dose.

The compounds of the invention are used, alone or in combination with one or more other active ingredients, to formulate pharmaceutical compositions of the invention. A pharmaceutical composition of the invention comprises: (a) an effective amount of a compound of Formula (I), or a pharmaceutically acceptable salt, pharmaceutically acceptable prodrug, or pharmaceutically active metabolite thereof; and (b) a pharmaceutically acceptable excipient.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of a compound of the invention and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the compounds of the invention may be prepared using suitable pharmaceutical excipients and compounding techniques now or later known or available to those skilled in the art. The compositions may be administered in the inventive methods by oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the compounds of the invention can be provided in the form of tablets or capsules, or as a solution, emulsion, or suspension. To prepare the oral compositions, the compounds may be formulated to yield a dosage of, e.g., from about 0.05 to about 100 mg/kg daily, or from about 0.05 to about 35 mg/kg daily, or from about 0.1 to about 10 mg/kg daily.

Oral tablets may include a compound according to the invention mixed with pharmaceutically acceptable excipients such as inert diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are suitable disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, compounds of the invention may be mixed with a solid, semi-solid, or liquid diluent. Soft gelatin capsules may be prepared by mixing the compound of the invention with water, an oil such as peanut oil, sesame oil, or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The compounds of this invention may also be administered by non-oral routes. For example, the compositions may be formulated for rectal administration as a suppository. For parenteral use, including intravenous, intramuscular, intraperitoneal, or subcutaneous routes, the compounds of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms will be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses may range from about 1 to 1000 μg/kg/minute of compound, admixed with a pharmaceutical carrier over a period ranging from several minutes to several days.

For topical administration, the compounds may be mixed with a pharmaceutical carrier at a concentration of about 0.1% to about 10% of drug to vehicle. Another mode of administering the compounds of the invention may utilize a patch formulation to affect transdermal delivery.

Compounds of the invention may alternatively be administered in methods of this invention by inhalation, via the nasal or oral routes, e.g., in a spray formulation also containing a suitable carrier.

Exemplary compounds useful in methods of the invention will now be described by reference to the illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I). Reactions may be performed between the melting point and the reflux temperature of the solvent, and preferably between 0° C. and the reflux temperature of the solvent.

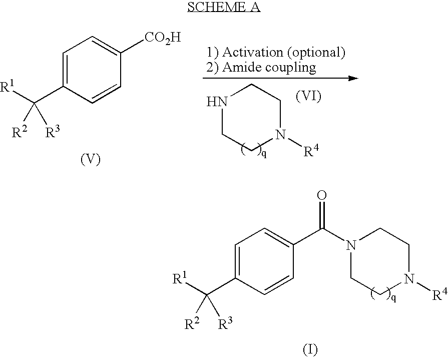

Compounds of Formula (I) may be prepared from benzoic acids (V), which are commercially available of available using methods known in the art. Coupling of acids (V) with amines (VI) is accomplished either directly using standard amide coupling methods, or by activating the acids to the corresponding acid chlorides and reacting the acid chlorides with amines (VI) in the presence of a suitable base, such as NaOH or Na₂CO₃, in a solvent such as toluene.

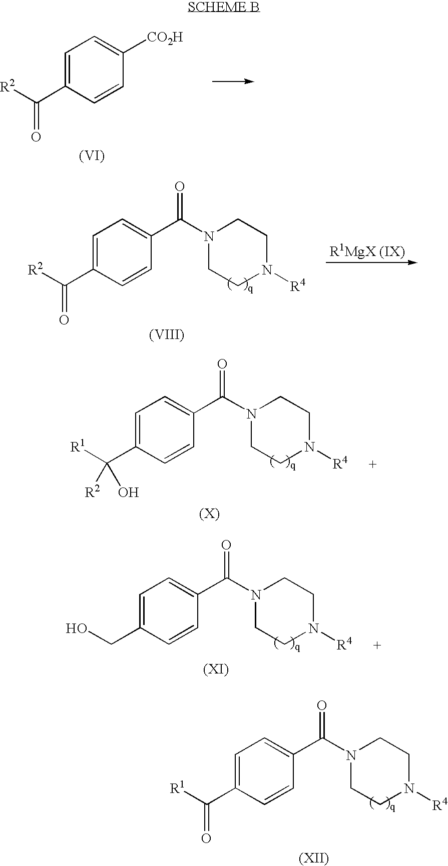

Benzoic acids (VI) are converted to amides (VIII) using coupling methods as described in Scheme A. Addition of suitable organometallic reagents, such as a Grignard reagents (IX), where X is Cl or Br, in a solvent such as tetrahydrofuran (THF) or diethyl ether (Et₂O), or a mixture thereof, provides alcohols (X). When R² is H, the reaction may also yield byproducts (XI) and (XII).

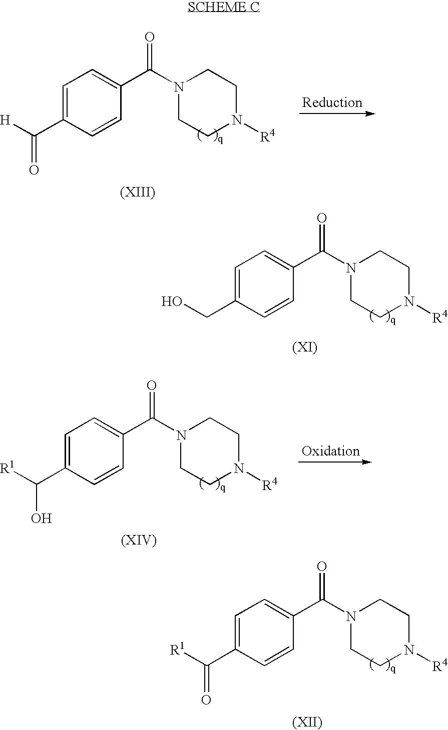

Alcohols (XI) and ketones (XII) are also available via reduction or oxidation protocols. Benzaldehydes (XIII) are reduced to provide benzyl alcohols (XI), using a suitable reducing agent such as NaBH₄ in a solvent such as methanol (MeOH). Secondary alcohols (XIV), available as in Scheme B where R² is H, may be oxidized using standard methods, such as Dess-Martin periodinane or Swern oxidation, to provide ketones (XII).

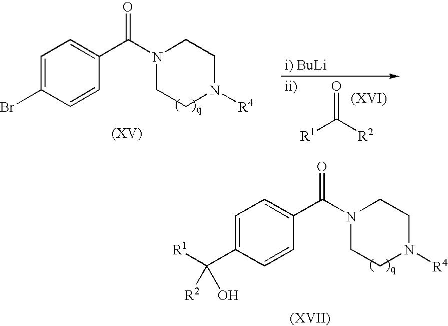

Amides (XV) are available from 4-bromobenzoic acid using the methods described in Scheme A. Halogen-metal exchange with a suitable organometallic reagent, such as BuLi, in a solvent such as THF, Et₂O, or a mixture thereof, followed by reaction with ketones (XVI) provides compounds of formula (XVII).

Those skilled in the art will recognize that several of the chemical transformations described above may be performed in a different order than that depicted in the above Schemes. In addition, one skilled in the art will recognize that compounds of formulae (X), (XI), (XII), (XIV), and (XVII) are compounds of Formula (I).

Compounds of Formula (I) may be converted to their corresponding salts using methods known to those skilled in the art. For example, amines of Formula (I) may be treated with trifluoroacetic acid (TFA), HCl, or citric acid in a solvent such as Et₂O, CH₂Cl₂, THF, or MeOH to provide the corresponding salt forms.

Compounds prepared according to the schemes described above may be obtained as single enantiomers, diastereomers, or regioisomers, by enantio-, diastero-, or regiospecific synthesis, or by resolution. Compounds prepared according to the schemes above may alternately be obtained as racemic (1:1) or non-racemic (not 1:1) mixtures or as mixtures of diastereomers or regioisomers. Where racemic and non-racemic mixtures of enantiomers are obtained, single enantiomers may be isolated using conventional separation methods known to one skilled in the art, such as chiral chromatography, recrystallization, diastereomeric salt formation, derivatization into diastereomeric adducts, biotransformation, or enzymatic transformation. Where regioisomeric or diastereomeric mixtures are obtained, single isomers may be separated using conventional methods such as chromatography or crystallization.

The following examples are provided to further illustrate the invention and various preferred embodiments.

### EXAMPLES

### Chemistry:

In obtaining the compounds described in the examples below and the corresponding analytical data, the following experimental and analytical protocols were followed unless otherwise indicated.

Unless otherwise stated, reaction mixtures were magnetically stirred at room temperature (rt) under a N_{2(g)} atmosphere. Where solutions are “dried,” they are generally dried over a drying agent such as Na₂SO₄ or MgSO₄. Where mixtures, solutions, and extracts were “concentrated”, they were typically concentrated on a rotary evaporator under reduced pressure.

Normal-phase flash column chromatography (FCC) was performed on silica gel (SiO₂) using prepackaged cartridges, unless otherwise indicated.

Analytical reversed-phase high performance liquid chromatography (HPLC) was performed on a Hewlett Packard HPLC Series 1100 with a Phenomenex Gemini C18 (5 μm, 4.6×150 mm) column. Detection was done at λ=220 and 254 nm. The gradient was either 1 to 99% acetonitrile with 20 mM aq. NH₄OH or with 0.5% TFA over 7.0 min with a flow rate of 1.5 mL/min. Preparative reversed-phase HPLC was performed on a Dionex APS2000 LC/MS with a Phenomenex Gemini C18 (5 μm, 30×100 mm) column with a gradient of acetonitrile in 20 mM aq. NH₄OH or on an Agilent Series 1100 preparative scale HPLC with a Phenomenex Gemini C18 (10 μm, 50×100 mm) column with a gradient of acetonitrile in 20 mM aq. NH₄OH.

Mass spectra (MS) were obtained on an Agilent series 1100 MSD using electrospray ionization (ESI) in positive mode unless otherwise indicated. Calculated (calcd.) mass corresponds to the exact mass.

Nuclear magnetic resonance (NMR) spectra were obtained on either a Bruker model DPX400 (400 MHz), DPX500 (500 MHz), or DRX600 (600 MHz) spectrometer. The format of the ¹H NMR data below is: chemical shift in ppm down field of the tetramethylsilane reference (multiplicity, coupling constant J in Hz, integration).

Chemical names were generated using ChemDraw Version 6.0.2 (CambridgeSoft, Cambridge, Mass.).

### Example 1

### [4-(Cyclohexyl-hydroxy-methyl)-phenyl]-(4-isopropyl-piperazin-1-yl)-methanone

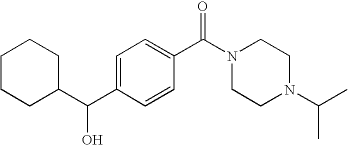

Step A; 4-(4-Isopropyl-piperazine-1-carbonyl)-benzaldehyde. To a suspension of 4-carboxybenzaldehyde (15.0 g, 100 mmol) in toluene (100 mL) at rt was added SOCl₂ (8.0 mL, 110 mmol) and N,N-dimethylformamide (DMF; 0.20 mL, 0.002 mmol). The flask was fitted with a reflux condenser, and the reaction was vented through 500 mL of 0.2 N NaOH to trap the evolved HCl and SO₂ gasses. The reaction was heated to 100° C. Vigorous gas evolution was observed. After 3 h at 100° C., the mixture was concentrated. The liquid residue was azeotroped with toluene (10 mL, 3×) to remove remaining SOCl₂. Crude 4-formyl-benzoyl chloride was obtained as a yellow liquid which solidified after storage at −20° C. To a solution of 4-formyl-benzoyl chloride (1.0 g, 5.9 mmol) in toluene (10 mL) at rt was added 10% aqueous (aq.) Na₂CO₃ (10 mL) and N-isopropylpiperazine (760 mg, 5.9 mmol). The biphasic mixture was stirred rapidly for 2 h. The layers were separated, and the aqueous layer was extracted with toluene. The combined organic layers were dried (Na₂SO₄) and concentrated to provide the title amide as an orange oil, which was used without further purification. ¹H NMR (rotameric broadening, CDCl₃): 10.05 (s, 1H), 7.93 (br d, J=8.0, 2H), 7.56 (br d, J=8.0, 2H), 3.95-3.70 (br m, 2H), 3.50-3.30 (br m, 2H), 2.77 (sept, J=6.6, 1H), 2.75-2.53 (br m, 2H), 2.53-2.37 (br m, 2H), 1.05 (d, J=6.5, 6H).

Step B; [4-(Cyclohexyl-hydroxy-methyl)-phenyl]-(4-isopropyl-piperazin-1-yl)-methanone. To a solution of 4-(4-isopropyl-piperazine-1-carbonyl)-benzaldehyde (351 mg, 1.35 mmol) in THF (15 mL) at −78° C. was added cyclohexylmagnesium chloride (2.0 M in Et₂O; 0.81 mL, 1.62 mmol). The mixture was allowed to warm to rt and stir for 5 h. The reaction was quenched with saturated (satd.) aq. NH₄Cl, poured into H₂O, and extracted with 3 portions of CH₂Cl₂. The combined organic layers were dried (Na₂SO₄) and concentrated. The crude residue was purified by preparative reversed-phase HPLC to provide the title amide as a viscous colorless liquid (129 mg, 28%). MS (ESI): mass calcd. for C₂₁H₃₂N₂O₂, 344.25; m/z found, 345 [M+H]⁺. HPLC: t_{R}=6.24 min. ¹H NMR (rotameric broadening, CDCl₃): 7.37 (br d, J=8.0, 2H), 7.32 (br d, J=8.2, 2H), 4.40 (d, J=6.9, 1H), 3.95-3.60 (br m, 2H), 3.60-3.25 (br m, 2H), 2.72 (sept, J=6.5, 1H), 2.65-2.30 (br m, 4H), 2.10-1.95 (br m, 1H), 1.95-1.88 (br m, 1H), 1.80-1.72 (br m, 1H), 1.72-1.55 (br m, 3H), 1.44-1.35 (br m, 1H), 1.28-0.85 (br m, 4H), 1.05 (d, J=6.5, 6H).

### Example 2

### [4-(1-Hydroxy-propyl)-phenyl]-(4-isopropyl-piperazin-1-yl)-methanone

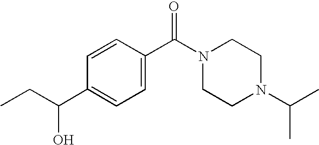

To a solution of 4-(4-isopropyl-piperazine-1-carbonyl)-benzaldehyde (200 mg, 0.77 mmol) in THF (5 mL) at rt was added ethylmagnesium bromide (1.0 M in Et₂O; 2.0 mL, 2.0 mmol). After 1 h, the mixture was quenched with satd. aq. NH₄Cl, poured into H₂O, and extracted with 3 portions of CH₂Cl₂. The combined organic layers were dried (Na₂SO₄) and concentrated. The crude residue was purified by preparative reversed-phase HPLC to provide the title amide as a viscous colorless liquid (112 mg, 50%). MS (ESI): mass calcd. for C₁₇H₂₆N₂O₂, 290.20; m/z found, 291 [M+H]⁺. HPLC: t_{R}=4.92 min. ¹H NMR (rotameric broadening, CDCl₃): 7.42-7.36 (m, 4H), 4.67-4.60 (m, 1H), 3.85-3.72 (br m, 2H), 3.52-3.35 (br m, 2H), 2.72 (sept, J=6.6, 1H), 2.68-2.34 (br m, 4H), 1.91-1.70 (m, 3H), 1.05 (d, J=6.5, 6H), 0.92 (t, J=7.4, 3H).

The compounds of Examples 3 and 4 were prepared by methods analogous to those described in EXAMPLE 2.

### Example 3

### [4-(Hydroxy-phenyl-methyl)-phenyl]-(4-isopropyl-piperazin-1-yl)-methanone

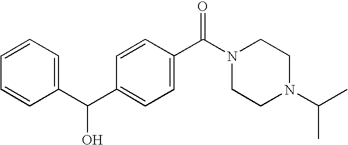

MS (ESI): mass calcd. for C₂₁H₂₆N₂O₂, 338.20; m/z found, 339 [M+H]⁺. HPLC: t_{R}=5.57 min. ¹H NMR (rotameric broadening, CDCl₃): 7.44-7.39 (m, 2H), 7.39-7.31 (m, 6H), 7.31-7.26 (m, 1H), 5.85 (s, 1H), 3.85-3.65 (br m, 2H), 3.55-3.30 (br m, 2H), 2.71 (sept, J=6.6, 1H), 2.64-2.32 (br m, 5H), 1.04 (d, J=6.6, 6H).

### Example 4

### [4-(1-Hydroxy-ethyl)-phenyl]-(4-isopropyl-piperazin-1-yl)-methanone

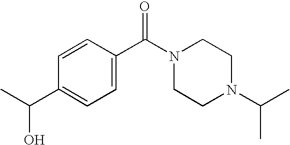

MS (ESI): mass calcd. for C₁₆H₂₄N₂O₂, 276.18; m/z found, 277 [M+H]⁺. HPLC: t_{R}=4.55 min. ¹H NMR (rotameric broadening, CDCl₃): 7.43-7.36 (m, 4H), 4.93 (q, J=6.4, 1H), 3.90-3.65 (br m, 2H), 3.60-3.35 (br m, 2H), 2.72 (sept, J=6.6, 1H), 2.68-2.35 (br m, 4H), 1.92 (br s, 1H), 1.50 (d, J=6.4, 3H), 1.05 (d, J=6.6, 6H).

### Example 5

### [4-(1-Hydroxy-2-methyl-propyl)-phenyl]-(4-isopropyl-piperazin-1-yl)-methanone

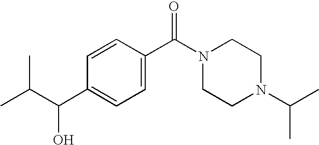

To a solution of 4-(4-isopropyl-piperazine-1-carbonyl)-benzaldehyde (286 mg, 1.1 mmol) in THF (10 mL) at rt was added isopropylmagnesium bromide (2.0 M in Et₂O; 1.1 mL, 2.2 mmol). After 10 min, the reaction was quenched with satd. aq. NH₄Cl, poured into H₂O, and extracted with 3 portions of CH₂Cl₂. The combined organic layers were dried (Na₂SO₄) and concentrated. The crude residue was purified by preparative reversed-phase HPLC to provide the title compound (131 mg, 39%). MS (ESI): mass calcd. for C₁₈H₂₈N₂O₂, 304.22; m/z found, 305 [M+H]⁺. HPLC: t_{R}=5.37 min. ¹H NMR (rotameric broadening, CDCl₃): 7.41-7.32 (m, 4H), 4.44-4.39 (m, 1H), 3.90-3.65 (br m, 2H), 3.55-3.30 (br m, 2H), 2.72 (sept, J=6.6, 1H), 2.67-2.34 (br m, 4H), 1.96 (oct, J=6.7, 1H), 1.87 (d, J=2.7, 1H), 1.05 (d, J=6.6, 6H), 0.98 (d, J=6.7, 3H), 0.82 (d, J=6.8, 3H).

### Example 6

### (4-Hydroxymethyl-phenyl)-(4-isopropyl-piperazin-1-yl)-methanone

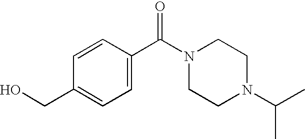

The title compound was obtained as a side-product of the reaction described in Example 5 (58 mg, 20%). MS (ESI): mass calcd. for C₁₅H₂₂N₂O₂, 262.17; m/z found, 263 [M+H]⁺. HPLC: t_{R}=4.33 min. ¹H NMR (rotameric broadening, CDCl₃): 7.43-7.37 (m, 4H), 4.73 (s, 2H), 3.90-3.60 (br m, 2H), 3.60-3.30 (br m, 2H), 2.72 (sept, J=6.6, 1H), 2.67-2.35 (br m, 4H), 1.87 (br s, 1H), 1.05 (d, J=6.6, 6H).

### Example 7

### [4-(Cyclohexyl-hydroxy-methyl)-phenyl]-(4-isopropyl-[1,4]diazepan-1-yl)-methanone

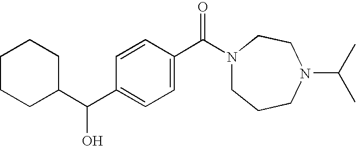

Step A; 1-Isopropyl-[1,4]diazepane. A solution of N-Boc-homopiperazine (20.0 g, 100 mmol), 1,2-dichloroethane (330 mL), and acetone (7.4 mL, 100 mmol) was stirred at rt and treated with NaBH(OAc)₃ (22.25 g, 105 mmol). After stirring overnight, the mixture washed twice with 100 mL 1 N NaOH. The organic layer was dried (Na₂SO₄) and concentrated to provide N-Boc-N′-isopropyl-homopiperazine as a pale yellow liquid, which was used without purification. ¹H NMR (CDCl₃): 3.50-3.36 (m, 4H), 2.90 (dsept, J=6.6, 1.6, 1H), 2.67-2.53 (m, 4H), 1.85-1.49 (m, 2H), 1.46 (s, 9H), 1.00 (d, J=6.6, 3H), 0.99 (d, J=6.6, 3H). The crude N-Boc-N′-isopropyl-homopiperazine was stirred rapidly in 1,4-dioxane (50 mL) at rt as HCl (4.0 M in dioxane; 125 mL) was added at a moderate rate producing a gummy precipitate. The mixture was heated to 45° C. and stirred for 6 h. The mixture was concentrated to provide the HCl salt as a viscous liquid. The crude salt was dissolved in H₂O (300 mL), made basic with NaOH (250 g), and extracted with CH₂Cl₂ (100 mL) five times. The combined organic layers were dried (Na₂SO₄) and concentrated to provide the free base of the title diazepane as a colorless liquid (11.71 g, 82%, 2 steps). ¹H NMR (CDCl₃): 2.97-2.85 (m, 5H), 2.70-2.62 (m, 4H), 2.25-2.08 (br m, 1H), 1.78-1.69 (m, 2H), 1.01 (d, J=6.6, 6H).

Step B; 4-(4-Isopropyl-[1,4]diazepane-1-carbonyl)-benzaldehyde. To a suspension of 4-carboxybenzaldehyde (15.0 g, 100 mmol) in toluene (100 mL) at rt was added SOCl₂ (8.0 mL, 110 mmol) and DMF (0.20 mL, 0.002 mmol). The flask was fitted with a reflux condenser, and the reaction was vented through 500 mL of 0.2 N NaOH to trap the evolved HCl and SO₂ gasses. After 3 h at 100° C., the homogeneous reaction mixture was concentrated. The liquid residue was azeotroped with toluene (3×10 mL) to remove remaining SOCl₂. Crude 4-formyl-benzoyl chloride was obtained as a yellow liquid which solidified after storage at −20° C. To a solution of 4-formyl-benzoyl chloride (2.0 g, 11.9 mmol) in toluene (15 mL) at rt was added 10% aq. Na₂CO₃ (15 mL) and 1-isopropyl-[1,4]diazepane (1.69 g, 11.9 mmol). The biphasic mixture was stirred rapidly for 3 h. The layers were separated, and the aqueous layer was extracted once with toluene. The combined organic layers were dried (Na₂SO₄) and concentrated to provide the title amide as an orange oil (2.99 g, 92%). This material was used without further purification. ¹H NMR (rotameric broadening, CDCl₃): 10.05 (s, 1H), 7.92 (br d, J=8.0, 2H), 7.55 (br d, J=8.0, 2H), 3.82-3.75 (br m, 2H), 3.42-3.34 (br m, 2H), 3.00-2.82 (br m, 1H), 2.82-2.77 (br m, 1H), 2.72-2.65 (br m, 1H), 2.65-2.54 (br m, 2H), 1.98-1.88 (br m, 1H), 1.75-1.64 (br m, 1H), 1.03 (d, J=6.6, 3H), 0.98 (d, J=6.6, 3H).

Step C. To a solution of 4-(4-isopropyl-[1,4]diazepane-1-carbonyl)-benzaldehyde (1.32 g, 4.8 mmol) in THF (30 mL) at rt was added cyclohexylmagnesium chloride (2.0 M in Et₂O; 4.8 mL, 9.6 mmol). The mixture was allowed to stir for 1 h at rt. The reaction was quenched with satd. aq. NH₄Cl, concentrated to remove THF, poured into H₂O, and extracted with 3 portions of CH₂Cl₂. The combined organic layers were dried (Na₂SO₄) and concentrated. The crude residue was purified by preparative reversed-phase HPLC to provide the title compound as a viscous colorless liquid (231 mg, 13%). MS (ESI): mass calcd. for C₂₂H₃₄N₂O₂, 358.26; m/z found, 359 [M+H]⁺. HPLC: t_{R}=6.67 min. ¹H NMR (rotameric broadening, CDCl₃): 7.36 (br d, J=8.1, 2H), 7.32 (br d, J=8.2, 2H), 4.41 (d, J=6.9, 1H), 3.79-3.72 (br m, 2H), 3.48-3.38 (br m, 2H), 3.00-2.82 (m, 1H), 2.82-2.75 (br m, 1H), 2.73-2.63 (br m, 1H), 2.63-2.54 (br m, 2H), 1.98-1.82 (br m, 3H), 1.82-1.55 (br m, 3H), 1.41 (br d, J=12.5, 1H), 1.28-1.11 (br m, 3H), 1.11-0.88 (br m, 2H), 1.03 (d, J=6.6, 3H), 0.98 (d, J=6.6, 3H).

### Example 8

### (4-Hydroxymethyl-phenyl)-(4-isopropyl-[1,4]diazepan-1-yl)-methanone

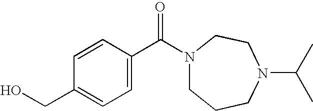

The title compound was obtained as a side-product from the reaction described in Example 7, Step C (106 mg, 8%). MS (ESI): mass calcd. for C₁₆H₂₄N₂O₂, 276.18; m/z found, 277 [M+H]⁺. HPLC: t_{R}=4.54 min. ¹H NMR (rotameric broadening, CDCl₃): 7.38 (br s, 4H), 4.72 (s, 2H), 3.78-3.72 (br m, 2H), 3.46-3.39 (br m, 2H), 3.00-2.82 (br m, 1H), 2.82-2.76 (br m, 1H), 2.71-2.65 (br m, 1H), 2.64-2.55 (br m, 2H), 1.95-1.88 (br m, 1H), 1.75-1.68 (br m, 1H), 1.03 (d, J=6.5, 3H), 0.98 (d, J=6.6, 3H).

### Example 9

### (4-Cyclohexanecarbonyl-phenyl)-(4-isopropyl-[1,4]diazepan-1-yl)-methanone

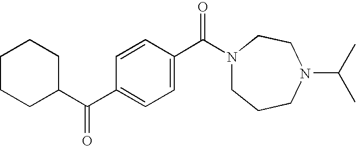

The title compound was obtained as a side-product from the reaction described in Example 7, Step C (221 mg, 13%). MS (ESI): mass calcd. for C₂₂H₃₂N₂O₂, 356.25; m/z found, 357 [M+H]⁺. HPLC: t_{R}=7.52 min. ¹H NMR (rotameric broadening, CDCl₃): 7.98-7.93 (m, 2H), 7.49-7.45 (m, 2H), 3.80-3.73 (br m, 2H), 3.41-3.34 (br m, 2H), 3.30-3.20 (m, 1H), 3.00-2.82 (br m, 1H), 2.81-2.76 (br m, 1H), 2.71-2.65 (br m, 1H), 2.65-2.54 (br m, 2H), 1.98-1.80 (br m, 5H), 1.80-1.64 (br m, 2H), 1.64-1.20 (br m, 5H), 1.03 (d, J=6.6, 3H), 0.98 (d, J=6.6, 3H).

### Example 10

### [4-(1-Hydroxy-propyl)-phenyl]-(4-isopropyl-[1,4]diazepan-1-yl)-methanone

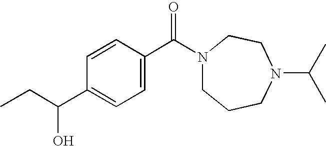

To a solution of 4-(4-isopropyl-[1,4]diazepane-1-carbonyl)-benzaldehyde (211 mg, 0.77 mmol) in THF (5 mL) at rt was added ethylmagnesium bromide (1.0 M in Et₂O; 2.0 mL, 2.0 mmol). After 1 h, the reaction was quenched with satd. aq. NH₄Cl, poured into H₂O, and extracted with 3 portions of CH₂Cl₂. The combined organic layers were dried (Na₂SO₄) and concentrated. The crude residue was purified by preparative reversed-phase HPLC to provide the title amide as a viscous colorless liquid (322 mg, 14%). MS (ESI): mass calcd. for C₁₈H₂₈N₂O₂, 304.22; m/z found, 305 [M+H]⁺. HPLC: t_{R}=5.19 min. ¹H NMR (rotameric broadening, CDCl₃): 7.40-7.33 (m, 4H), 4.63 (t, J=6.5, 1H), 3.79-3.72 (br m, 2H), 3.46-3.39 (br m, 2H), 3.00-2.82 (m, 1H), 2.79 (br t, J=5.1, 1H), 2.68 (br t, J=5.7, 1H), 2.63-2.54 (m, 2H), 2.00-1.68 (br m, 5H), 1.03 (d, J=6.6, 3H), 0.98 (d, J=6.6, 3H), 0.92 (t, J=7.4, 3H).

The compounds of Examples 11-13 were prepared by methods analogous to those described in EXAMPLE 10.

### Example 11

### [4-(Hydroxy-phenyl-methyl)-phenyl]-(4-isopropyl-[1,4]diazepan-1-yl)-methanone

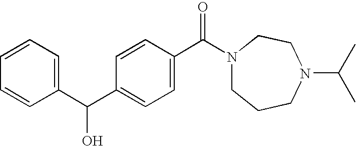

MS (ESI): mass calcd. for C₂₂H₂₈N₂O₂, 352.22; m/z found, 353 [M+H]⁺. HPLC: t_{R}=5.90 min. ¹H NMR (rotameric broadening, CDCl₃): 7.43-7.31 (m, 8H), 7.31-7.25 (m, 1H), 5.85 (s, 1H), 3.77-3.71 (br m, 2H), 3.45-3.38 (br m, 2H), 2.98-2.82 (m, 1H), 2.78 (br t, J=5.1, 1H), 2.67 (br t, J=5.7, 1H), 2.62-2.52 (m, 2H), 2.49-2.30 (br m, 1H), 1.95-1.85 (br m, 1H), 1.75-1.65 (br m, 1H), 1.02 (d, J=6.6, 3H), 0.97 (d, J=6.6, 3H).

### Example 12

### [4-(1-Hydroxy-ethyl)-phenyl]-(4-isopropyl-[1,4]diazepan-1-yl)-methanone

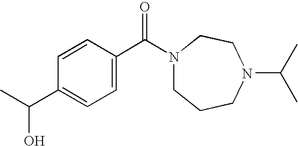

MS (ESI): mass calcd. for C₁₇H₂₆N₂O₂, 290.20; m/z found, 291 [M+H]⁺. HPLC: t_{R}=4.79 min. ¹H NMR (rotameric broadening, CDCl₃): 7.42-7.35 (m, 4H), 4.93 (q, J=6.4, 1H), 3.80-3.70 (br m, 2H), 3.48-3.39 (br m, 2H), 3.00-2.82 (m, 1H), 2.79 (br t, J=5.2, 1H), 2.68 (br t, J=5.8, 1H), 2.65-2.55 (m, 2H), 1.96-1.86 (br m, 1H), 1.90-1.80 (br s, 1H), 1.78-1.68 (br m, 1H), 1.50 (d, J=6.5, 3H), 1.03 (d, J=6.6, 3H), 0.98 (d, J=6.6, 3H).

### Example 13

### [4-(1-Hydroxy-2-methyl-propyl)-phenyl]-(4-isopropyl-[1,4]diazepan-1-yl)-methanone

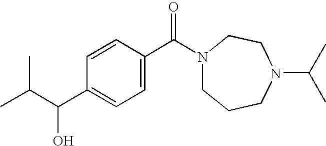

MS (ESI): mass calcd. for C₁₉H₃₀N₂O₂, 318.23; m/z found, 319 [M+H]⁺. HPLC: t_{R}=5.72 min. ¹H NMR (rotameric broadening, CDCl₃): 7.41-7.31 (m, 4H), 4.41 (d, J=6.6, 1H), 3.80-3.73 (br m, 2H), 3.47-3.39 (br m, 2H), 3.00-2.82 (br m, 1H), 2.82-2.76 (br m, 1H), 2.68 (br t, J=5.7, 1H), 2.65-2.56 (br m, 2H), 1.97 (oct, J=6.7, 1H), 1.95-1.86 (br m, 2H), 1.75-1.68 (br m, 1H), 1.03 (d, J=6.6, 3H), 0.98 (dd, J=6.7, 2.0, 6H), 0.82 (d, J=6.8, 3H).

### Example 14

### (4-cyclobutyl-piperazin-1-yl)-[4-(hydroxy-phenyl-methyl)-phenyl]-methanone

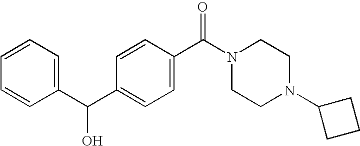

Step A; 1-Cyclobutyl-piperazine bis-hydrochloride. A solution of N-Boc-piperazine (25.0 g, 134 mmol), 1,2-dichloroethane (425 mL), and cyclobutanone (9.4 g, 134 mmol) was stirred at rt for 45 min, cooled in an ice bath to 10° C., and then treated with NaBH(OAc)₃ (28.43 g, 134 mmol). The mixture was allowed to warm to rt and was stirred overnight. The resulting cloudy reaction mixture washed with 1 N NaOH (2×75 mL). The organic layer was dried (Na₂SO₄) and concentrated to provide N-Boc-N′-cyclobutylpiperazine as a pale yellow liquid. ¹H NMR (DMSO-d₆): 3.50-3.40 (m, 4H), 2.75-2.67 (m, 1H), 2.32-2.21 (m, 4H), 2.07-2.00 (m, 2H), 1.92-1.82 (m, 2H), 1.76-1.67 (m, 2H), 1.46 (s, 9H). The unpurified N-Boc-N′-cyclobutylpiperazine was stirred rapidly in 1,4-dioxane (67 mL) at rt as HCl (4.0 M in 1,4-dioxane; 133 mL) was added at a moderate rate producing a white precipitate. The suspension was heated to 50° C. and stirred for 6 h. The mixture was cooled to 0° C., and hexane (125 mL) was added to assist precipitation of the bis-hydrochloride salt. The precipitate was collected by suction filtration, washed with hexane, and air dried to provide the desired hydrochloride salt as a white powder (27.09 g, 95%, 2 steps). ¹H NMR (DMSO-d₆): 12.32 (br s, 1H), 9.70 (br s, 2H), 3.80-3.65 (br m, 1H), 3.62-3.30 (br m, 6H), 3.20-2.95 (br m, 2H), 2.44-2.28 (br m, 2H), 2.22-2.12 (br m, 2H), 1.80-1.72 (m, 1H), 1.72-1.62 (m, 1H).

Step B; 4-(4-Cyclobutyl-piperazine-1-carbonyl)-benzaldehyde. To a suspension of 4-carboxybenzaldehyde (15.0 g, 100 mmol) in toluene (100 mL) at rt was added SOCl₂ (8.0 mL, 110 mmol) and DMF (0.20 mL, 0.002 mmol). The flask was fitted with a reflux condenser, and the reaction was vented through 500 mL of 0.2 N NaOH to trap the evolved HCl and SO₂ gasses. After 3 h at 100° C., the homogeneous reaction mixture was concentrated. The liquid residue was azeotroped with toluene (3×10 mL) to remove remaining SOCl₂. The crude 4-formylbenzoyl chloride was obtained as a yellow liquid which solidified after storage at −20° C. To a solution of 4-formylbenzoyl chloride (2.0 g, 11.9 mmol) in toluene (15 mL) at rt was added 10% aq. Na₂CO₃ (15 mL) and 1-cyclobutyl-piperazine bis-hydrochloride (2.54 g, 11.9 mmol). The biphasic mixture was stirred rapidly for 3 h. The layers were separated, and the aqueous layer was extracted with toluene. The combined organic layers were dried (Na₂SO₄) and concentrated to provide the crude amide as an orange oil which was purified by FCC (2 M N₃ in MeOH/ethyl acetate (EtOAc)). The title amide was obtained as a pale yellow viscous liquid (2.93 g, 90%). ¹H NMR (rotameric broadening, CDCl₃): 10.05 (s, 1H), 7.95-7.91 (m, 2H), 7.57-7.53 (m, 2H), 3.90-3.70 (br m, 2H), 3.50-3.30 (br m, 2H), 2.81-2.70 (m, 1H), 2.50-2.30 (br m, 4H), 2.08-2.00 (br m, 2H), 1.95-1.80 (br m, 2H), 1.80-1.63 (br m, 2H).

Step C; (4-Cyclobutyl-piperazin-1-yl)-[4-(hydroxy-phenyl-methyl)-phenyl]-methanone. To a solution of 4-(4-cyclobutyl-piperazine-1-carbonyl)-benzaldehyde (300 mg, 1.1 mmol) in THF (10 mL) at rt was added phenylmagnesium bromide (1.0 M in THF, 2.2 mL, 2.2 mmol). After 10 min, the reaction was quenched with satd. aq. NH₄Cl, concentrated to remove THF, poured into H₂O, and extracted with 2 portions of CH₂Cl₂. The combined organic layers were dried (Na₂SO₄) and concentrated. The crude residue was purified by preparative reversed-phase HPLC to provide the title amide as a viscous colorless liquid (267 mg, 69%). MS (ESI): mass calcd. for C₂₂H₂₆N₂O₂, 350.20; m/z found, 351 [M+H]⁺. HPLC: t_{R}=5.81 min. ¹H NMR (rotameric broadening, CDCl₃): 7.45-7.40 (br m, 2H), 7.40-7.31 (br m, 6H), 7.31-7.25 (br m, 1H), 5.86 (d, J=2.9, 1H), 3.90-3.70 (br m, 2H), 3.60-3.30 (br m, 2H), 2.79-2.69 (m, 1H), 2.46-2.13 (br m, 4H), 2.28 (d, J=3.4, 1H), 2.07-1.98 (br m, 2H), 1.93-1.78 (br m, 2H), 1.78-1.62 (br m, 2H).

The compounds of Examples 15-16 were prepared by methods analogous to those described in EXAMPLE 14.

### Example 15

### (4-Cyclobutyl-piperazin-1-yl)-[4-(1-hydroxy-propyl)-phenyl]-methanone

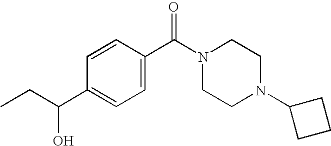

MS (ESI): mass calcd. for C₁₈H₂₆N₂O₂, 302.20; m/z found, 303 [M+H]⁺. HPLC: t_{R}=5.17 min. ¹H NMR (rotameric broadening, CDCl₃): 7.41-7.33 (m, 4H), 4.63 (br t, J=6.2, 1H), 3.90-3.65 (br m, 2H), 3.60-3.30 (br m, 2H), 2.80-2.70 (m, 1H), 2.50-2.14 (br m, 4H), 2.10-1.98 (br m, 3H), 1.94-1.56 (m, 6H), 0.92 (t, J=7.4, 3H).

### Example 16

### (4-Cyclobutyl-piperazin-1-yl)-[4-(1-hydroxy-2-methyl-propyl)-phenyl]-methanone

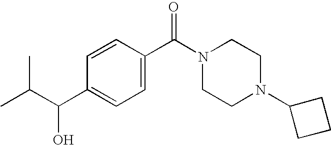

MS (ESI): mass calcd. for C₁₉H₂₈N₂O₂, 316.22; m/z found, 317 [M+H]⁺.

HPLC: t_{R}=5.59 min. ¹H NMR (rotameric broadening, CDCl₃): 7.40-7.32 (m, 4H), 4.42 (dd, J=6.6, 3.2, 1H), 3.90-3.60 (br m, 2H), 3.60-3.30 (br m, 2H), 2.80-2.70 (br m, 1H), 2.50-2.15 (br m, 4H), 2.10-1.80 (m, 6H), 1.80-1.63 (m, 2H), 0.98 (d, J=6.7, 3H), 0.82 (d, J=6.8, 3H).

### Example 17

### (4-Cyclobutyl-piperazin-1-yl)-[4-(cyclohexyl-hydroxy-methyl)-phenyl]-methanone

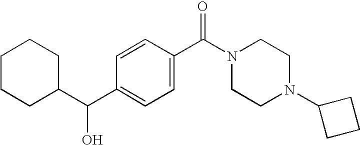

To a solution of 4-(4-cyclobutyl-piperazine-1-carbonyl)-benzaldehyde (300 mg, 1.1 mmol) in THF (10 mL) at rt was added cyclohexylmagnesium bromide (2.0 M in Et₂O; 1.1 mL, 2.2 mmol). After 10 min, the reaction was quenched with satd. aq. NH₄Cl, concentrated to remove THF, poured into H₂O, and extracted with 2 portions of CH₂Cl₂. The combined organic layers were dried (Na₂SO₄) and concentrated. The crude residue was purified by preparative reversed-phase HPLC to provide the title compound (48 mg, 12%). MS (ESI): mass calcd. for C₂₂H₃₂N₂O₂, 356.25; m/z found, 357 [M+H]⁺. HPLC: t_{R}=6.54 min. ¹H NMR (rotameric broadening, CDCl₃): 7.40-7.30 (m, 4H), 4.41 (d, J=6.8, 1H), 3.94-3.65 (br m, 2H), 3.60-3.30 (br m, 2H), 2.80-2.70 (m, 1H), 2.50-2.10 (br m, 4H), 2.09-1.95 (br m, 2H), 1.95-1.76 (br m, 4H), 1.82-1.50 (br m, 6H), 1.44-1.35 (br m, 1H), 1.30-0.85 (m, 5H).

### Example 18

### (4-Cyclobutyl-piperazin-1-yl)-(4-hydroxymethyl-phenyl)-methanone

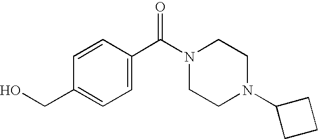

The title compound was obtained as a side-product from the reaction described in Example 17 (60 mg, 20%). MS (ESI): mass calcd. for C₁₆H₂₂N₂O₂, 274.17; m/z found, 275 [M+H]⁺. HPLC: t_{R}=4.57 min. ¹H NMR (rotameric broadening, CDCl₃): 7.43-7.35 (m, 4H), 4.73 (s, 2H), 3.90-3.60 (br m, 2H), 3.60-3.30 (br m, 2H), 2.80-2.70 (m, 1H), 2.50-2.12 (br m, 4H), 2.10-1.99 (br m, 2H), 1.98-1.78 (br m, 3H), 1.80-1.62 (br m, 2H).

### Example 19

### (4-Cyclobutyl-[1,4]diazepan-1-yl)-[4-(1-hydroxy-propyl)-phenyl]-methanone

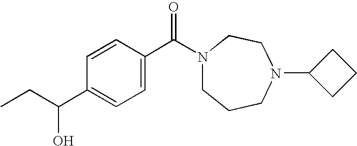

Step A; 1-Cyclobutyl-[1,4]diazepane bis-hydrochloride. A solution of N-Boc-homopiperazine (20.00 g, 99.8 mmol), 1,2-dichloroethane (400 mL), and cyclobutanone (6.99 g, 99.8 mmol) was stirred at rt for 45 min, cooled in an ice bath to 10° C., and then treated with NaBH(OAc)₃ (21.17 g, 99.8 mmol). The mixture was allowed to warm to rt and was stirred overnight. The resulting cloudy reaction mixture washed with 1 N NaOH (2×75 mL). The organic layer was dried (Na₂SO₄) and concentrated to provide N-Boc-N′-cyclobutyl-homopiperazine as a pale yellow liquid. The unpurified N-Boc-N′-cyclobutyl-homopiperazine was stirred rapidly in 1,4-dioxane (50 mL) at rt as HCl (4.0 M in 1,4-dioxane; 100 mL) was added at a moderate rate producing a white precipitate. The suspension was heated to 50° C. and stirred for 6 h. The mixture was cooled to 0° C., and hexane (125 mL) was added to assist precipitation of the bis-hydrochloride salt. The precipitate was collected by suction filtration, washed with hexane, and air dried to provide the desired hydrochloride salt as a white powder (18.57 g, 82%, 2 steps). ¹H NMR (DMSO-d₆): 11.92 (br s, 1H), 9.87 (br s, 1H), 9.45 (br s, 1H), 3.78-3.68 (m, 1H), 3.67-3.58 (br m, 1H), 3.58-3.47 (br m, 2H), 3.47-3.34 (br m, 2H), 3.34-3.28 (br m, 1H), 3.28-3.14 (br m, 1H), 3.09-3.00 (br m, 1H), 2.38 (quint, J=10.0, 2H), 2.24-2.18 (br m, 4H), 1.75-1.67 (m, 1H), 1.67-1.57 (m, 1H).

Step B; 4-(4-Cyclobutyl-[1,4]diazepane-1-carbonyl)-benzaldehyde. To a suspension of 4-carboxybenzaldehyde (15.0 g, 100 mmol) in toluene (100 mL) at rt was added SOCl₂ (8.0 mL, 110 mmol) and DMF (0.20 mL, 0.002 mmol). The flask was fitted with a reflux condenser, and the reaction was vented through 500 mL of 0.2 N NaOH to trap the evolved HCl and SO₂ gasses. After 3 h at 100° C., the homogeneous reaction mixture was concentrated. The liquid residue was azeotroped with toluene (3×10 mL) to remove remaining SOCl₂. The crude 4-formylbenzoyl chloride was obtained as a yellow liquid which solidified after storage at −20° C. To a solution of 4-formylbenzoyl chloride (2.0 g, 11.9 mmol) in toluene (15 mL) at rt was added 10% aq. Na₂CO₃ (15 mL) and 1-cyclobutyl-[1,4]diazepane bis-hydrochloride (2.70 g, 11.9 mmol). The biphasic mixture was stirred rapidly for 3 h. The layers were separated, and the aqueous layer was extracted with toluene. The combined organic layers were dried (Na₂SO₄) and concentrated to provide the crude amide as an orange oil which was purified by FCC (2 M N₃ in MeOH/EtOAc). The title amide was obtained as a pale yellow viscous liquid (2.37 g, 70%). ¹H NMR (rotameric broadening, CDCl₃): 10.05 (s, 1H), 7.95-7.91 (m, 2H), 7.57-7.53 (m, 2H), 3.83-3.76 (br m, 2H), 3.45-3.37 (br m, 2H), 2.99-2.81 (m, 1H), 2.66-2.61 (br m, 1H), 2.55-2.50 (br m, 1H), 2.48-2.38 (br m, 2H), 2.02-1.82 (br m, 3H), 1.82-1.52 (br m, 5H).

Step C; (4-Cyclobutyl-[1,4]diazepan-1-yl)-[4-(1-hydroxy-propyl)-phenyl]-methanone. To a solution of 4-(4-cyclobutyl-[1,4]diazepane-1-carbonyl)-benzaldehyde (315 mg, 1.1 mmol) in THF (10 mL) at rt was added ethylmagnesium bromide (1.0 M in THF; 2.2 mL, 2.2 mmol). After 10 min, the reaction was quenched with satd. aq. NH₄Cl, concentrated to remove THF, poured into H₂O, and extracted with two portions of CH₂Cl₂. The combined organic layers were dried (Na₂SO₄) and concentrated. The crude residue was purified by preparative reversed-phase HPLC to provide the title amide as a viscous colorless liquid (168 mg, 48%). MS (ESI): mass calcd. for C₁₉H₂₈N₂O₂, 316.22; m/z found, 317 [M+H]⁺. HPLC: t_{R}=5.30 min. ¹H NMR (rotameric broadening, CDCl₃): 7.37 (br s, 4H), 4.63 (br t, J=6.3, 1H), 3.81-3.73 (br m, 2H), 3.52-3.41 (br m, 2H), 2.96-2.79 (m, 1H), 2.65-2.59 (br m, 1H), 2.54-2.47 (br m, 1H), 2.47-2.37 (br m, 2H), 2.10-1.90 (br m, 3H), 1.90-1.55 (br m, 7H), 0.92 (t, J=7.4, 3H).

The compounds of Examples 20-22 were prepared by methods analogous to those described in EXAMPLE 19.

### Example 20

### (4-Cyclobutyl-[1,4]diazepan-1-yl)-[4-(cyclohexyl-hydroxy-methyl)-phenyl]-methanone

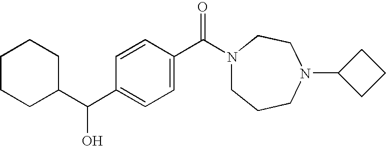

MS (ESI): mass calcd. for C₂₃H₃₄N₂O₂, 370.26; m/z found, 371 [M+H]⁺. HPLC: t_{R}=6.72 min. ¹H NMR (rotameric broadening, CDCl₃): 7.39-7.30 (m, 4H), 4.45-4.37 (m, 1H), 3.81-3.73 (br m, 2H), 3.52-3.41 (br m, 2H), 2.96-2.90 (m, 1H), 2.67-2.60 (br m, 1H), 2.54-2.48 (br m, 1H), 2.47-2.27 (br m, 2H), 2.12-1.85 (br m, 4H), 1.88-1.70 (br m, 5H), 1.72-1.52 (br m, 4H), 1.45-1.36 (br m, 1H), 1.33-0.85 (m, 5H).

### Example 21

### (4-Cyclobutyl-[1,4]diazepan-1-yl)-[4-(hydroxy-phenyl-methyl)-phenyl]-methanone

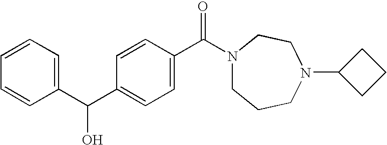

MS (ESI): mass calcd. for C₂₃H₂₈N₂O₂, 364.22; m/z found, 365 [M+H]⁺. HPLC: t_{R}=5.95 min. ¹H NMR (rotameric broadening, CDCl₃): 7.45-7.31 (m, 8H), 7.31-7.25 (m, 1H), 5.86 (s, 1H), 3.81-3.70 (br m, 2H), 3.50-3.39 (br m, 2H), 2.95-2.78 (m, 1H), 2.65-2.56 (br m, 1H), 2.54-2.45 (br m, 1H), 2.44-2.35 (br m, 2H), 2.12-1.91 (br m, 3H), 1.91-1.55 (br m, 6H).

### Example 22

### (4-Cyclobutyl-[1,4]diazepan-1-yl)-[4-(1-hydroxy-2-methyl-propyl)-phenyl]-methanone

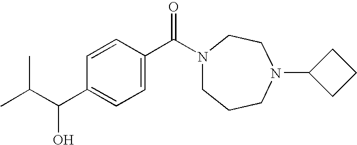

MS (ESI): mass calcd. for C₂₀H₃₀N₂O₂, 330.23; m/z found, 331 [M+H]⁺. HPLC: t_{R}=5.74 min. ¹H NMR (rotameric broadening, CDCl₃): 7.40-7.31 (m, 4H), 4.41 (dd, J=6.6, 2.8, 1H), 3.81-3.74 (br m, 2H), 3.52-3.41 (br m, 2H), 2.98-2.80 (m, 1H), 2.65-2.60 (br m, 1H), 2.56-2.50 (br m, 1H), 2.47-2.39 (br m, 2H), 2.10-1.90 (m, 4H), 1.90-1.55 (m, 6H), 0.98 (d, J=6.7, 3H), 0.82 (d, J=6.8, 3H).

### Example 23

### (4-Cyclobutyl-[1,4]diazepan-1-yl)-(4-hydroxymethyl-phenyl)-methanone

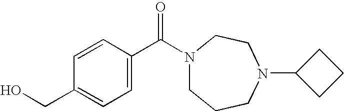

To a solution of 4-(4-cyclobutyl-[1,4]diazepane-1-carbonyl)-benzaldehyde (200 mg, 0.70 mmol) in MeOH (5 mL) at rt was added NaBH₄ (26 mg, 0.70 mmol). After 2 h, the reaction was poured into satd. aq. NaHCO₃ and concentrated. The residue was diluted with H₂O and extracted with CH₂Cl₂ (3×). The combined organic layers were dried (Na₂SO₄) and concentrated. The crude residue was purified by preparative reversed-phase HPLC to provide the title amide as a viscous colorless liquid (162 mg, 80%). MS (ESI): mass calcd. for C₁₇H₂₄N₂O₂, 288.18; m/z found, 289 [M+H]⁺. HPLC: t_{R}=4.68 min.

¹H NMR (rotameric broadening, CDCl₃): 7.42-7.33 (m, 4H), 4.72 (s, 2H), 3.82-3.74 (br m, 2H), 3.50-3.40 (br m, 2H), 2.97-2.79 (m, 1H), 2.65-2.59 (br m, 1H), 2.55-2.47 (br m, 1H), 2.47-2.37 (br m, 2H), 2.12-1.90 (br m, 3H), 1.93-1.51 (br m, 6H).

### Example 24

### [4-(Cyclohexyl-hydroxy-methyl)-phenyl]-(4-cyclopropyl-piperazin-1-yl)-methanone

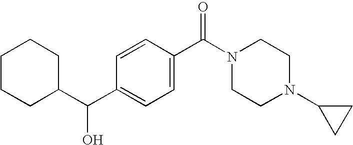

Step A; 1-Cyclopropyl-piperazine bis-hydrochloride. A solution of N-Boc-piperazine (29.82 g, 160 mmol), 1:1 THF/MeOH (300 mL), (1-ethoxycyclopropoxy)-trimethylsilane (64 mL, 320 mmol), acetic acid (15 mL, 262 mmol), and NaBH₃CN (15.10 g, 240 mmol) was stirred at 50° C. for 5 h. The reaction was cooled to rt and quenched by addition of H₂O (15 mL). After 5 min, 1 N NaOH (60 mL) was added, and the mixture was stirred for 15 min. The mixture was concentrated removing the bulk of the THF and MeOH. The residue was diluted with CH₂Cl₂ (300 mL) and washed with 1 N NaOH (300 mL). The aqueous layer was back-extracted once with CH₂Cl₂, and the combined organic layers were washed with satd. aq. NaCl (2×300 mL), dried (Na₂SO₄) and concentrated to provide N-Boc-N′-cyclopropyl-piperazine as white solid. ¹H NMR (CDCl₃): 3.39 (br t, J=5.0, 4H), 2.55 (br t, J=4.6, 4H), 1.64-1.56 (m, 1H), 1.46 (s, 9H), 0.50-0.38 (m, 4H). The unpurified N-Boc-N′-cyclopropyl-piperazine was stirred rapidly in 1,4-dioxane (75 mL) at rt as HCl (4.0 M in 1,4-dioxane; 195 mL) was added at a moderate rate. A very thick suspension formed immediately but thinned as more HCl in 1,4-dioxane was added. The suspension was heated to 45° C. and stirred for 6 h. The mixture was cooled to rt, and the precipitated product was collected by suction filtration, washed with 1,4-dioxane, and dried under vacuum to provide the desired hydrochloride salt as a white powder (28.71 g, 90%, 2 steps). ¹H NMR (CD₃OD): 9.90-9.40 (br s, 2H), 3.80-3.20 (br m, 9H), 1.20-0.90 (br m, 2H), 0.85-0.58 (br m, 2H).

Step B; 4-(4-Cyclopropyl-piperazine-1-carbonyl)-benzaldehyde. To a suspension of 4-carboxybenzaldehyde (5.0 g, 33.3 mmol) in toluene (50 mL) at rt was added SOCl₂ (2.9 mL, 40 mmol) and DMF (0.20 mL, 0.002 mmol). The flask was fitted with a reflux condenser, and the reaction was vented through 500 mL of 0.2 N NaOH to trap the evolved HCl and SO₂ gasses. After 3 h at 100° C., the homogeneous reaction mixture was concentrated. The liquid residue was azeotroped with toluene (3×10 mL) to remove remaining SOCl₂. The crude 4-formylbenzoyl chloride was obtained as a yellow liquid, which solidified after storage at −20° C. To a solution of 4-formylbenzoyl chloride (33.3 mmol) in toluene (15 mL) at rt was added water (40 mL), NaOH (4.72 g, 118 mmol), and 1-cyclopropyl-piperazine bis-hydrochloride (6.37 g, 32 mmol). The biphasic mixture was stirred rapidly for 3 h. The layers were separated, and the aqueous layer was extracted with toluene. The combined organic layers were dried (Na₂SO₄) and concentrated to provide the amide as an orange oil, which was used without further purification (7.69 g, 93%). ¹H NMR (CDCl₃): 10.06 (s, 1H), 7.97-7.90 (m, 2H), 7.59-7.53 (m, 2H), 3.82-3.66 (br m, 2H), 3.43-3.25 (br m, 2H), 2.77-2.48 (br m, 4H), 1.68-1.62 (m, 1H), 0.51-0.47 (br m, 4H).

Step C; [4-(Cyclohexyl-hydroxy-methyl)-phenyl]-(4-cyclopropyl-piperazin-1-yl)-methanone. To a solution of 4-(4-cyclopropyl-piperazine-1-carbonyl)-benzaldehyde (284 mg, 1.1 mmol) in THF (10 mL) at rt was added cyclohexylmagnesium chloride (2.0 M in Et₂O; 1.1 mL, 2.2 mmol). After 10 min, the reaction was quenched with satd. aq. NH₄Cl, concentrated to remove THF, poured into H₂O, and extracted with 2 portions of CH₂Cl₂. The combined organic layers were dried (Na₂SO₄) and concentrated. The crude residue was purified by preparative reversed-phase HPLC to provide the title amide as a viscous colorless liquid (35 mg, 9%). MS (ESI): mass calcd. for C₂₁H₃₀N₂O₂, 342.23; m/z found, 343 [M+H]⁺. HPLC: t_{R}=6.38 min. ¹H NMR (rotameric broadening, CDCl₃): 7.40-7.35 (m, 2H), 7.35-7.31 (m, 2H), 4.42 (d, J=6.9, 1H), 3.86-3.60 (br m, 2H), 3.50-3.25 (br m, 2H), 2.80-2.44 (br m, 4H), 1.98-1.83 (br m, 2H), 1.83-1.72 (br m, 1H), 1.72-1.52 (br m, 4H), 1.45-1.38 (br m, 1H), 1.29-0.88 (br m, 5H), 0.52-0.45 (m, 2H), 0.45-0.39 (m, 2H).

The compounds of Examples 25-26 were prepared by methods analogous to those described in EXAMPLE 24.

### Example 25

### (4-Cyclopropyl-piperazin-1-yl)-[4-(hydroxy-phenyl-methyl)-phenyl]-methanone

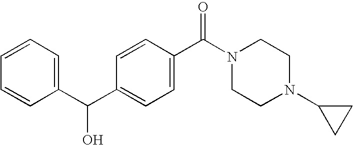

MS (ESI): mass calcd. for C₂₁H₂₄N₂O₂, 336.18; m/z found, 337 [M+H]⁺. HPLC: t_{R}=5.67 min. ¹H NMR (rotameric broadening, CDCl₃): 7.45-7.40 (m, 2H), 7.45-7.31 (m, 6H), 7.31-7.26 (m, 1H), 5.86 (s, 1H), 3.85-3.60 (br m, 2H), 3.50-3.25 (br m, 2H), 2.80-2.45 (br m, 4H), 1.67-1.58 (m, 1H), 0.52-0.38 (m, 4H).

### Example 26

### (4-Cyclopropyl-piperazin-1-yl)-[4-(1-hydroxy-2-methyl-propyl)-phenyl]-methanone

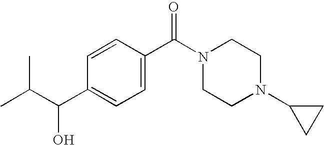

MS (ESI): mass calcd. for C₁₈H₂₆N₂O₂, 302.20; m/z found, 303 [M+H]⁺. HPLC: t_{R}=5.45 min. ¹H NMR (rotameric broadening, CDCl₃): 7.41-7.32 (m, 4H), 4.42 (dd, J=6.6, 3.2, 1H), 3.87-3.60 (br m, 2H), 3.57-3.25 (br m, 2H), 2.80-2.45 (br m, 4H), 1.96 (oct, J=6.7, 1H), 1.88 (d, J=3.4, 1H), 1.69-1.60 (m, 1H), 0.99 (d, J=6.7, 3H), 0.82 (d, J=6.8, 3H), 0.51-0.38 (m, 4H).

### Example 27

### [4-(Cyclohexyl-hydroxy-methyl)-phenyl]-(4-cyclopropyl-[1,4]diazepan-1-yl)-methanone

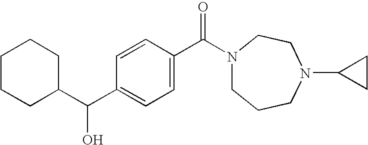

Step A; 1-Cyclopropyl-[1,4]diazepane bis-hydrochloride. A solution of N-Boc-homopiperazine (25.09 g, 125 mmol), 1:1 THF/MeOH (230 mL), (1-ethoxycyclo-propoxy)trimethylsilane (50 mL, 250 mmol), acetic acid (11.5 mL, 200 mmol), and NaBH₃CN (11.8 g, 188 mmol) was stirred at 50° C. for 5 h. The reaction was cooled to rt and quenched by addition of H₂O (15 mL). After 5 min, 1 N NaOH (50 mL) was added, and the mixture was stirred 15 min. The mixture was concentrated removing the bulk of the THF and MeOH. The residue was diluted with CH₂Cl₂ (300 mL) and washed with 1 N NaOH (300 mL). The aqueous layer was back-extracted once with CH₂Cl₂, and the combined organic layers were washed with satd. aq. NaCl (2×300 mL), dried (Na₂SO₄) and concentrated to provide N-Boc-N′-cyclopropyl-homopiperazine as white solid. ¹H NMR (CDCl₃): 3.52-3.42 (br m, 2H), 3.46-3.38 (br m, 2H), 2.84-2.72 (br m, 4H), 1.86-1.72 (br m, 3H), 1.46 (s, 9H), 0.49-0.42 (br m, 2H), 0.42-0.35 (br m, 2H). The unpurified N-Boc-N′-cyclopropyl-homopiperazine was stirred rapidly in 1,4-dioxane (60 mL) at rt as HCl (4.0 M in 1,4-dioxane; 150 mL) was added at a moderate rate. Some precipitate formed during the addition. The thin suspension was heated to 45° C. and stirred for 6 h. The mixture was concentrated, and the solids were resuspended in 1:1 1,4-dioxane/hexanes and cooled to 0° C. The solid product was collected by suction filtration, washed with 1:1 1,4-dioxane/hexanes, and dried under vacuum to provide the desired hydrochloride salt as a white powder (25.34 g, 95%, 2 steps). ¹H NMR (CD₃OD): 9.76-9.55 (br s, 1H), 9.46-9.25 (br s, 1H), 3.85-3.48 (br m, 5H), 3.48-3.32 (br m, 3H), 3.06-2.90 (br m, 1H), 2.22-2.10 (br m, 2H), 1.24-1.08 (br m, 2H), 0.90-0.75 (br m, 2H).

Step B; 4-Formyl-benzoyl chloride. To a suspension of 4-carboxybenzaldehyde (10.5 g, 70.0 mmol) in toluene (100 mL) at rt was added SOCl₂ (9.2 g, 77.3 mmol) and DMF (1.0 mL, 0.013 mmol). After heating at ca. 75° C. for 6 h, the mixture was cooled to rt. The resulting acid chloride solution was used in the next step without further manipulation.

Step C; 4-(4-Cyclopropyl-[1,4]diazepane-1-carbonyl)-benzaldehyde. A solution of 1-cyclopropyl-[1,4]diazepane dihydrochloride salt (14.0 g, 65.7 mmol) and 1 N NaOH (200 mL) in toluene (100 mL) was stirred at 0° C. for 0.5 h, then was treated, over 40 min, with the solution of 4-formyl-benzoyl chloride prepared in Step B. The reaction mixture was stirred at 0° C. for 1 h and subsequently at rt for 16 h. The reaction mixture was basified with 1 N NaOH (pH 12) and the phases were separated. The aqueous layer was extracted with EtOAc (3×50 mL). The organic layers were pooled, dried (MgSO₄), filtered, and concentrated to afford the crude product as a viscous reddish-brown oil (19.8 g, 94%). MS (ESI): mass calcd. for C₁₆H₂₀N₂O₂, 272.15; m/z found, 273.1 [M+H]⁺. ¹H NMR (CDCl₃): 10.0 (s, 1H), 7.92 (pseudo d, 2H, J=9.7), 7.54 (pseudo d, 2H, J=9.1), 3.77 (br s, 2H), 3.40 (br s, 2H), 2.99 (m, 1H), 2.82-2.65 (m, 2H), 2.00-1.74 (m, 4H), 0.54-0.36 (m, 4H).

Step D. To a solution of 4-(4-cyclopropyl-[1,4]diazepane-1-carbonyl)-benzaldehyde (300 mg, 1.1 mmol) in THF (10 mL) at rt was added cyclohexyl-magnesium chloride (2.0 M in Et₂O; 1.1 mL, 2.2 mmol). After 10 min, the reaction was quenched with satd. aq. NH₄Cl, concentrated to remove THF, poured into H₂O, and extracted with 2 portions of CH₂Cl₂. The combined organic layers were dried (Na₂SO₄) and concentrated. The crude residue was purified by preparative reversed-phase HPLC to provide the title compound (59 mg, 15%). MS (ESI): mass calcd. for C₂₂H₃₂N₂O₂, 356.25; m/z found, 357 [M+H]⁺. HPLC: t_{R}=6.52 min. ¹H NMR (rotameric broadening, CDCl₃): 7.39-7.30 (m, 4H), 4.41 (d, J=6.9, 1H), 3.80-3.71 (br m, 2H), 3.51-3.40 (br m, 2H), 2.99-2.93 (m, 1H), 2.88-2.82 (br m, 1H), 2.81-2.73 (br m, 2H), 1.99-1.72 (br m, 6H), 1.72-1.53 (br m, 3H), 1.45-1.37 (br m, 1H), 1.28-1.01 (br m, 4H), 1.01-0.89 (br m, 1H), 0.52-0.33 (br m, 4H).

### Example 28

### (4-Cyclopropyl-[1,4]diazepan-1-yl)-(4-hydroxymethyl-phenyl)-methanone

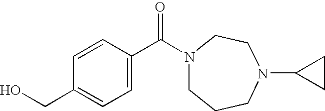

The title compound was obtained as a side product from the reaction described in Example 27, Step D (72 mg, 24%). MS (ESI): mass calcd. for C₁₆H₂₂N₂O₂, 274.17; m/z found, 275 [M+H]⁺. HPLC: t_{R}=4.47 min. ¹H NMR (rotameric broadening, CDCl₃): 7.41-7.35 (m, 4H), 4.72 (s, 2H), 3.80-3.74 (m, 2H), 3.47-3.40 (m, 2H), 3.00-2.94 (br m, 1H), 2.88-2.82 (br m, 1H), 2.81-2.72 (br m, 2H), 2.00-1.80 (br m, 2H), 1.80-1.70 (m, 1H), 0.53-0.40 (m, 3H), 0.40-0.34 (m, 1H).

### Example 29

### (4-Cyclohexanecarbonyl-phenyl)-(4-cyclopropyl-[1,4]diazepan-1-yl)-methanone

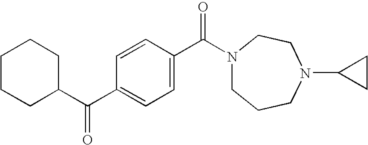

The title compound was obtained as a side product from the reaction described in Example 27, Step D (54 mg, 14%). MS (ESI): mass calcd. for C₂₂H₃₀N₂O₂, 354.23; m/z found, 355 [M+H]⁺. HPLC: t_{R}=7.27 min. ¹H NMR (rotameric broadening, CDCl₃): 7.99-7.94 (m, 2H), 7.48-7.44 (m, 2H), 3.80-3.72 (br m, 2H), 3.42-3.35 (br m, 2H), 3.30-3.20 (br m, 1H), 3.00-2.93 (m, 1H), 2.90-2.83 (br m, 1H), 2.82-2.72 (br m, 2H), 1.98-1.80 (m, 6H), 1.80-1.70 (m, 2H), 1.57-1.20 (m, 5H), 0.54-0.40 (m, 3H), 0.40-0.35 (m, 1H).

The compounds of Examples 30-32 were prepared by methods analogous to those described in EXAMPLE 27.

### Example 30

### (4-Cyclopropyl-[1,4]diazepan-1-yl)-[4-(hydroxy-phenyl-methyl)-phenyl]-methanone

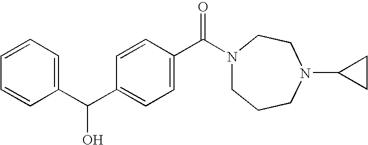

MS (ESI): mass calcd. for C₂₂H₂₆N₂O₂, 350.20; m/z found, 351 [M+H]⁺.

HPLC: t_{R}=5.77 min. ¹H NMR (rotameric broadening, CDCl₃): 7.44-7.40 (m, 2H), 7.40-7.32 (m, 5H), 7.31-7.25 (m, 1H), 5.86 (s, 1H), 3.79-3.73 (br m, 2H), 3.50-3.41 (br m, 2H), 2.99-2.93 (m, 1H), 2.87-2.81 (m, 1H), 2.81-2.72 (br m, 2H), 2.32 (br s, 1H), 1.99-1.72 (m, 3H), 0.53-0.41 (m, 3H), 0.41-0.33 (m, 1H).

### Example 31

### (4-Cyclopropyl-[1,4]diazepan-1-yl)-[4-(1-hydroxy-propyl)-phenyl]-methanone

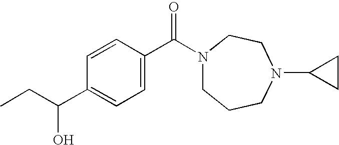

MS (ESI): mass calcd. for C₁₈H₂₆N₂O₂, 302.20; m/z found, 303 [M+H]⁺. HPLC: t_{R}=5.11 min. ¹H NMR (rotameric broadening, CDCl₃): 7.40-7.34 (m, 4H), 4.63 (t, J=6.4, 1H), 3.79-3.73 (br m, 2H), 3.50-3.41 (br m, 2H), 2.98-2.92 (br m, 1H), 2.86 (br t, J=5.5, 1H), 2.82-2.72 (br m, 2H), 2.00-1.70 (br m, 6H), 0.92 (t, J=7.4, 3H), 0.52-0.35 (m, 4H).

### Example 32

### (4-Cyclopropyl-[1,4]diazepan-1-yl)-[4-(1-hydroxy-2-methyl-propyl)-phenyl]-methanone

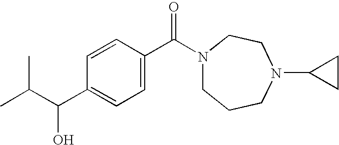

MS (ESI): mass calcd. for C₁₉H₂₈N₂O₂, 316.22; m/z found, 317 [M+H]⁺. HPLC: t_{R}=5.56 min. ¹H NMR (rotameric broadening, CDCl₃): 7.39-7.31 (m, 4H), 4.41 (dd, J=6.6, 3.0, 1H), 3.79-3.72 (br m, 2H), 3.49-3.40 (br m, 2H), 2.99-2.93 (br m, 1H), 2.89-2.82 (br m, 1H), 2.82-2.73 (br m, 2H), 2.00-1.70 (br m, 5H), 0.98 (d, J=6.6, 3H), 0.82 (d, J=6.8, 3H), 0.54-0.33 (m, 4H).

### Example 33

### (4-tert-Butyl-phenyl)-(4-cyclobutyl-piperazin-1-yl)-methanone

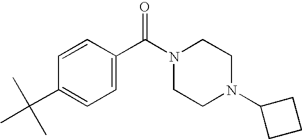

To a solution of 4-tert-butyl benzoic acid (168 mg, 0.94 mmol) and 4-cyclobutyl piperazine bis-hydrochloride (200 mg, 0.94 mmol) in DMF (2.8 mL) was added K₂CO₃ (260 mg, 1.9 mmol), 1-hydroxybenzotriazole (190 mg, 1.4 mmol) and 1-(3-dimethylaminopropyl)-3-ethylcarbodiimide hydrochloride (272 mg, 1.4 mmol). After 24 h, the reaction mixture was partitioned between EtOAc and 1 N NaOH (20 mL). The organic layer washed with satd. aq. NaCl, dried (MgSO₄) and concentrated. The resulting residue was purified by FCC (MeOH/CH₂Cl₂) to provide 256 mg (91%) of the title compound. MS (ESI): mass calcd. for C₁₉H₂₈N₂O, 300.45; m/z found, 301.2 [M+H]⁺. ¹H NMR (CDCl₃): 7.40 (d, J=8.5, 2H), 7.33 (d, J=8.5, 2H), 3.79 (br s, 2H), 3.47 (br s, 2H), 2.74 (p, J=8.0, 1H), 2.38 (br s, 2H), 2.26 (br s, 2H), 2.06-2.00 (m, 2H), 1.91-1.83 (m, 2H), 1.76-1.66 (m, 2H), 1.32 (s, 9H).

The compounds of Examples 34-40 were prepared using methods analogous to that described in Example 33.

### Example 34

### (4-Cyclobutyl-piperazin-1-yl)-(4-ethyl-phenyl)-methanone

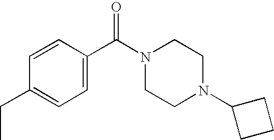

MS (ESI): mass calcd. for C₁₇H₂₄N₂O, 272.39; m/z found, 273.2 [M+H]⁺.

### Example 35

### (4-Cyclobutyl-piperazin-1-yl)-(4-isopropyl-phenyl)-methanone

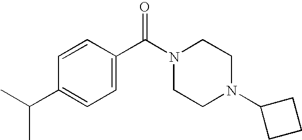

MS (ESI): mass calcd. for C₁₈H₂₆N₂O, 286.42; m/z found, 387.2 [M+H]⁺.

### Example 36

### (4-Cyclobutyl-piperazin-1-yl)-(4-cyclohexyl-phenyl)-methanone

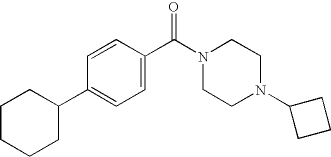

MS (ESI): mass calcd. for C₂₁H₃₀N₂O, 326.49; m/z found, 327.3 [M+H]⁺.

### Example 37

### (4-Benzyl-phenyl)-(4-cyclobutyl-piperazin-1-yl)-methanone

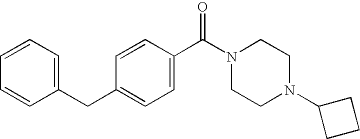

MS (ESI): mass calcd. for C₂₂H₂₆N₂O, 334.47; m/z found, 335.2 [M+H]⁺.

### Example 38

### (4-Cyclobutyl-piperazin-1-yl)-(4-propyl-phenyl)-methanone

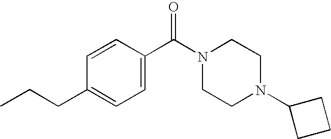

MS (ESI): mass calcd. for C₁₈H₂₆N₂O, 286.42; m/z found, 287.2 [M+H]⁺.

### Example 39

### (4-Butyl-phenyl)-(4-cyclobutyl-piperazin-1-yl)-methanone

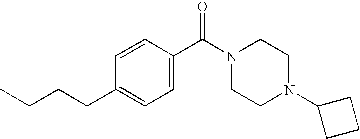

MS (ESI): mass calcd. for C₁₉H₂₈N₂O, 300.45; m/z found, 301.3 [M+H]⁺.

### Example 40

### (4-Cyclobutyl-piperazin-1-yl)-(4-pentyl-phenyl)-methanone

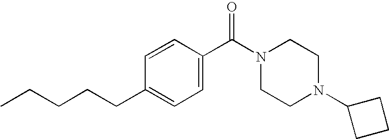

MS (ESI): mass calcd. for C₂₀H₃₀N₂O, 314.47; m/z found, 315.3 [M+H]⁺.

### Example 41

### (4-Cyclobutyl-piperazin-1-yl)-[4-(1-hydroxy-1-methyl-ethyl)-phenyl]-methanone

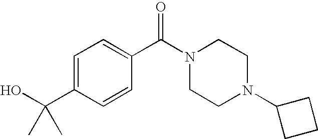

Step A; (4-Bromo-phenyl)-(4-cyclobutyl-piperazin-1-yl)-methanone. A solution of 4-bromobenzoic acid (2.0 g, 9.9 mmol), benzotriazol-1-yl-oxytripyrrolidinophosphonium hexafluorophosphate (7.7 g, 14.9 mmol) and 1-hydroxybenzotriazole (2.0 g, 14.9 mmol) in CH₂Cl₂ (100 mL) was treated with 1-cyclobutyl-piperazine bis-hydrochloride (2.5 g, 11.9 mmol) followed by Et₃N (4.0 g, 39.8 mmol). After 24 h, the mixture was diluted with water (250 mL) and extracted with CH₂Cl₂ (3×100 mL). The combined organic layers were dried and concentrated. The crude oil was purified by reverse phase chromatography to give the title compound (1.9 g). MS (ESI): mass calcd. for C₁₅H₁₉BrN₂O, 322.1; m/z found, 323.1 [M+H]⁺. ¹H NMR (DMSO-d₆): 7.56 (d, J=8.5 Hz, 2H), 7.30 (d, J=8.5 Hz, 2H), 3.86-3.72 (m, 2H), 3.49-3.36 (m, 2H), 2.79-2.73 (m, 1H), 2.44-2.35 (m, 2H), 2.33-2.20 (m, 2H), 2.09-2.01 (m, 2H), 1.92-1.84 (m, 2H), 1.78-1.68 (m, 2H).

Step B; (4-Cyclobutyl-Piperazin-1-yl)-[4-(1-hydroxy-1-methyl-ethyl)-phenyl]-methanone. To a −78° C. solution of (4-bromo-phenyl)-(4-cyclobutyl-piperazin-1-yl)-methanone (55 mg, 0.17 mmol) in THF (2.0 mL) was added dropwise a solution of n-BuLi (1.6 M in hexanes; 0.22 mL, 0.35 mmol). After 10 min, acetone (11 mg, 0.19 mmol) was added and the reaction was allowed to warm to rt. After 2 h at rt, the reaction was quenched with satd. aq. NH₄Cl (10 mL) and extracted with EtOAc (2×10 mL). The combined organic layers were dried and concentrated. Purification of the crude residue by reverse phase chromatography gave the title compound (2.0 mg). MS (ESI): mass calcd. for C₁₈H₂₆N₂O₂, 302.2; m/z found, 303.2 [M+H]⁺. ¹H NMR (DMSO-d₆): 7.52 (d, J=8.5 Hz, 2H), 7.37 (d, J=8.5 Hz, 2H), 3.86-3.71 (m, 2H), 3.52-3.38 (m, 2H), 2.78-2.71 (m, 1H), 2.45-2.33 (m, 2H), 2.31-2.19 (m, 2H), 2.08-1.98 (m, 2H), 1.92-1.81 (m, 2H), 1.77-1.64 (m, 3H), 1.58 (s, 6H).

### Example 42

### (4-Cyclobutyl-piperazin-1-yl)-[4-(1-hydroxy-cyclohexyl)-phenyl]-methanone

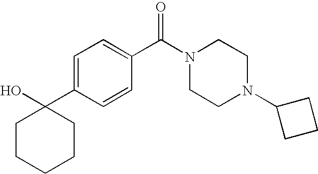

The title compound was prepared using methods analogous to those described in Example 41. MS (ESI): mass calcd. for C₂₁H₃₀N₂O₂, 342.2; m/z found, 343.2 [M+H]⁺. ¹H NMR (CDCl₃): 7.55 (d, J=8.5 Hz, 2H), 7.40 (d, J=8.5 Hz, 2H), 3.85-3.76 (m, 2H), 3.50-3.42 (m, 2H), 2.80-2.74 (m, 1H), 2.46-2.36 (m, 2H), 2.33-2.22 (m, 2H), 2.09-2.02 (m, 2H), 1.93-1.64 (m, 13H), 1.36-1.26 (m, 2H).

The compounds in Examples 43-47 are prepared using methods analogous to those described in the preceding examples.

### Example 43

### (4-Cyclopropyl-[1,4]diazepan-1-yl)-[4-(1-hydroxy-cyclohexyl)-phenyl]-methanone

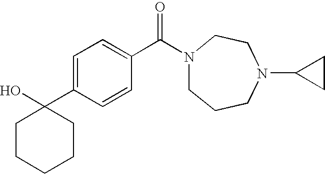

### Example 44

### (4-Cyclopropyl-[1,4]diazepan-1-yl)-[4-(1-hydroxy-cyclopentyl)-phenyl]-methanone

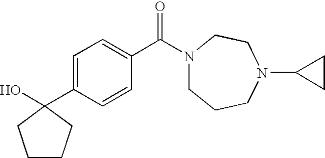

### Example 45

### (4-Cyclobutyl-piperazin-1-yl)-[4-(1-hydroxy-cyclopentyl)-phenyl]-methanone

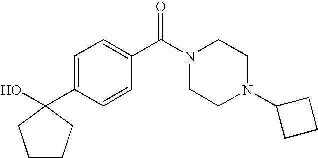

### Example 46

### (4-Cyclopropyl-[1,4]diazepan-1-yl)-[4-(1-hydroxy-cycloheptyl)-phenyl]-methanone

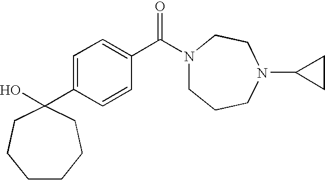

### Example 47

### [4-(1-Hydroxy-cycloheptyl)-phenyl]-(4-isopropyl-piperazin-1-yl)-methanone

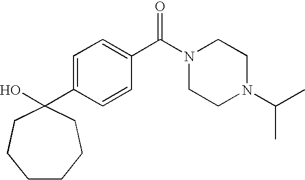

The compounds in Examples 48-51 were prepared using methods analogous to those described in the preceding examples.

### Example 48

### (4-Cyclopropyl-piperazin-1-yl)-[4-(1-hydroxy-propyl)-phenyl]-methanone

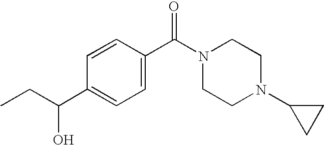

MS (ESI): mass calcd. for C₁₇H₂₄N₂O₂, 288.18; m/z found, 289 [M+H]⁺.

¹H NMR (rotameric broadening, CDCl₃): 7.42-7.35 (m, 4H), 4.64 (t, J=6.6 Hz, 1H), 3.88-3.60 (bm, 2H), 3.50-3.25 (br m, 2H), 2.80-2.45 (br m, 4H), 2.10-1.85 (m, 1H), 1.85-1.67 (m, 2H), 1.66-1.54 (m, 1H), 0.93 (t, J=7.4 Hz, 3H), 0.52-0.45 (m, 2H), 0.45-0.39 (m, 2H).

### Example 49

### (4-Cyclopropyl-piperazin-1-yl)-(4-hydroxymethyl-phenyl)-methanone

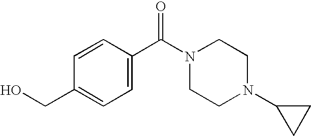

MS (ESI): mass calcd. for C₁₅H₂₀N₂O₂, 260.15; m/z found, 261 [M+H]⁺. ¹H NMR (rotameric broadening, CDCl₃): 7.39 (bs, 4H), 4.72 (s, 2H), 3.90-3.60 (br m, 2H), 3.60-3.20 (br m, 2H), 2.80-2.40 (br m, 4H), 2.30-1.80 (br m, 1H), 1.67-1.59 (m, 1H), 0.52-0.45 (m, 2H), 0.45-0.38 (m, 2H).

### Example 50

### (4-Butyl-piperazin-1-yl)-(4-hydroxymethyl-phenyl)-methanone

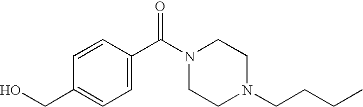

MS (ESI): mass calcd. for C₁₆H₂₄N₂O₂, 276.18; m/z found, 277.2 [M+H]⁺. ¹H NMR (CDCl₃): 7.43-7.33 (m, 4H), 4.71 (s, 2H), 3.84-3.70 (m, 2H), 3.51-3.32 (m, 2H), 2.58-2.27 (m, 5H), 2.20-2.11 (m, 4H), 1.54-1.18 (m, 4H), 0.90 (t, J=7.3, 3H).

### Example 51

### (4-sec-Butyl-piperazin-1-yl)-(4-hydroxymethyl-phenyl)-methanone

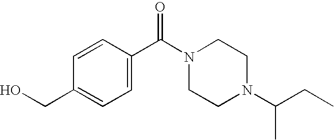

MS (ESI): mass calcd. for C₁₆H₂₄N₂O₂, 276.18; m/z found, 277.2 [M+H]⁺. ¹H NMR (CDCl₃): 7.38-7.34 (m, 4H), 4.70 (s, 2H), 3.92-3.61 (m, 2H), 3.51-3.25 (m, 2H), 2.69-2.18 (m, 6H), 1.62-1.43 (m, 1H), 1.37-1.16 (m, 1H), 0.96 (d, J=6.5, 3H), 0.89 (t, J=7.3, 3H).

### Biological Methods:

### H₃ Receptor Binding

Binding of compounds to the cloned human and rat H₃ receptors, stably expressed in SK-N-MC cells, was performed as described by Barbier, A. J. et al. (Br. J. Pharmacol. 2004, 143(5), 649-661). Data for compounds tested in these assays are presented in Table 1 (human) and Table 2 (rat), as an average of the results obtained.

TABLE 1

| | Human H₃ |
|---|---|
| Ex. | Kᵢ (nM) |
| 1 | 7 |
| 2 | 32 |
| 3 | 14 |
| 4 | 58 |
| 5 | 6 |
| 6 | 164 |
| 7 | 2 |
| 8 | 31 |
| 9 | 3 |
| 10 | 9 |
| 11 | 2 |
| 12 | 17 |
| 13 | 6 |
| 14 | 4 |
| 15 | 12 |
| 16 | 15 |
| 17 | 4 |
| 18 | 43 |
| 19 | 1 |
| 20 | 1 |
| 21 | 1 |
| 22 | 1 |
| 23 | 2 |
| 24 | 19 |
| 25 | 59 |
| 26 | 84 |
| 27 | 1 |
| 28 | 20 |
| 29 | 1 |
| 30 | 2 |
| 31 | 4 |
| 32 | 3 |
| 33 | 5 |
| 34 | 28 |
| 35 | 11 |
| 36 | 9 |
| 37 | 7 |
| 38 | 31 |
| 39 | 24 |
| 40 | 17 |
| 41 | 32 |
| 42 | 7 |
| 48 | 221 |
| 49 | 586 |
| 50 | 55 |
| 51 | 530 |

TABLE 2

| | Rat H₃ |
|---|---|
| Ex. | Kᵢ (nM) |
| 7 | 75 |
| 10 | 509 |
| 11 | 78 |
| 27 | 37 |
| 30 | 28 |
| 31 | 205 |
| 33 | 152 |
| 36 | 79 |

### Cyclic AMP Accumulation

Sublines of SK-N-MC cells were created that expressed a reporter construct and either the human or rat H₃ receptor. The pA₂ values were obtained as described by Barbier et al. (2004). Data for compounds tested in these assays are presented in Table 3, as an average of the results obtained (NT=not tested).

TABLE 3

| | Human | Rat |
|---|---|---|
| Ex. | pA₂ | pA₂ |
| 6 | NT | 6.53 |
| 7 | 9.06 | 7.76 |
| 19 | 9.74 | 8.48 |
| 27 | 9.02 | 8.16 |
| 29 | 9.04 | 8.16 |
| 30 | 9.38 | 8.82 |
| 32 | 9.10 | 7.92 |
| 33 | 8.44 | 7.87 |

What is claimed is:

1. A compound of Formula (I): 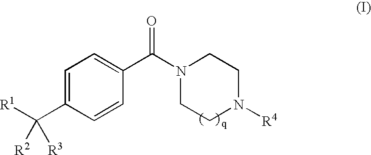
wherein
R¹ is H, C₁₋₄alkyl, monocyclic C₃₋₇cycloalkyl, or phenyl;
R² is H or methyl;
or R¹ and R² taken together form monocyclic C₃₋₇cycloalkyl;
R³ is H, OH, or methyl;
or, when R¹ is not H or phenyl, R² and R³ taken together form a carbonyl;
q is 1 or 2; and
R⁴ is —C₂₋₆alkyl, —C₃₋₆alkenyl, —C₃₋₆alkynyl, monocyclic cycloalkyl, or —C₁₋₂alkyl-(monocyclic cycloalkyl), each unsubstituted or substituted with —OH, —OC₁₋₄alkyl, fluoro, —NH₂, —NH(C₁₋₄alkyl), or —N(C₁₋₄alkyl)₂;
provided that when R¹ is phenyl, and R² and R³ are both H, then q is 1;
or a pharmaceutically acceptable salt, a pharmaceutically acceptable prodrug,
or a pharmaceutically active metabolite thereof.

2. A compound as defined in claim 1, wherein R¹ is H, methyl, ethyl, propyl, isopropyl, butyl, cyclohexyl, or phenyl.

3. A compound as defined in claim 1, wherein R² is H.

4. A compound as defined in claim 1, wherein R¹ and R² taken together form cyclohexyl.

5. A compound as defined in claim 1, wherein R³ is OH.

6. A compound as defined in claim 2, wherein R³ is OH.

7. A compound as defined in claim 4, wherein R³ is OH.

8. A compound as defined in claim 1, wherein R⁴ is ethyl, propyl, isopropyl, sec-butyl, cyclopropyl, cyclobutyl, cyclopentyl, cyclohexyl, cyclopropylmethyl, cyclobutylmethyl, or cyclopentylmethyl, each unsubstituted or substituted as previously described.

9. A compound as defined in claim 1, wherein R⁴ is isopropyl, cyclopropyl, or cyclobutyl.

10. A compound as defined in claim 5, wherein R⁴ is isopropyl, cyclopropyl, or cyclobutyl.

11. A compound as defined in claim 7, wherein R⁴ is isopropyl, cyclopropyl, or cyclobutyl.

12. A compound as defined in claim 1, wherein R¹ is H or C₁₋₆alkyl, R² is H, R³ is H or methyl, and R⁴ is cyclopropyl or cyclobutyl.

13. A compound selected from the group consisting of:
[4-(Cyclohexyl-hydroxy-methyl)-phenyl]-(4-isopropyl-piperazin-1-yl)-methanone;
[4-(1-Hydroxy-propyl)-phenyl]-(4-isopropyl-piperazin-1-yl)-methanone;
[4-(Hydroxy-phenyl-methyl)-phenyl]-(4-isopropyl-piperazin-1-yl)-methanone;
[4-(1-Hydroxy-ethyl)-phenyl]-(4-isopropyl-piperazin-1-yl)-methanone;
[4-(1-Hydroxy-2-methyl-propyl)-phenyl]-(4-isopropyl-piperazin-1-yl)-methanone;
(4-Hydroxymethyl-phenyl)-(4-isopropyl-piperazin-1-yl)-methanone;
[4-(Cyclohexyl-hydroxy-methyl)-phenyl]-(4-isopropyl-[1,4]diazepan-1-yl)-methanone;
(4-Hydroxymethyl-phenyl)-(4-isopropyl-[1,4]diazepan-1-yl)-methanone;
(4-Cyclohexanecarbonyl-phenyl)-(4-isopropyl-[1,4]diazepan-1-yl)-methanone;
[4-(1-Hydroxy-propyl)-phenyl]-(4-isopropyl-[1,4]diazepan-1-yl)-methanone;
[4-(Hydroxy-phenyl-methyl)-phenyl]-(4-isopropyl-[1,4]diazepan-1-yl)-methanone;
[4-(1-Hydroxy-ethyl)-phenyl]-(4-isopropyl-[1,4]diazepan-1-yl)-methanone;
[4-(1-Hydroxy-2-methyl-propyl)-phenyl]-(4-isopropyl-[1,4]diazepan-1-yl)-methanone;
(4-Cyclobutyl-piperazin-1-yl)-[4-(hydroxy-phenyl-methyl)-phenyl]-methanone;
(4-Cyclobutyl-piperazin-1-yl)-[4-(1-hydroxy-propyl)-phenyl]-methanone;
(4-Cyclobutyl-piperazin-1-yl)-[4-(1-hydroxy-2-methyl-propyl)-phenyl]-methanone;
(4-Cyclobutyl-piperazin-1-yl)-[4-(cyclohexyl-hydroxy-methyl)-phenyl]-methanone;
(4-Cyclobutyl-piperazin-1-yl)-(4-hydroxymethyl-phenyl)-methanone;
(4-Cyclobutyl-[1,4]diazepan-1-yl)-[4-(1-hydroxy-propyl)-phenyl]-methanone;
(4-Cyclobutyl-[1,4]diazepan-1-yl)-[4-(cyclohexyl-hydroxy-methyl)-phenyl]-methanone;
(4-Cyclobutyl-[1,4]diazepan-1-yl)-[4-(hydroxy-phenyl-methyl)-phenyl]-methanone;
(4-Cyclobutyl-[1,4]diazepan-1-yl)-[4-(1-hydroxy-2-methyl-propyl)-phenyl]-methanone;
(4-Cyclobutyl-[1,4]diazepan-1-yl)-(4-hydroxymethyl-phenyl)-methanone;
[4-(Cyclohexyl-hydroxy-methyl)-phenyl]-(4-cyclopropyl-piperazin-1-yl)-methanone;
(4-Cyclopropyl-piperazin-1-yl)-[4-(hydroxy-phenyl-methyl)-phenyl]-methanone;
(4-Cyclopropyl-piperazin-1-yl)-[4-(1-hydroxy-2-methyl-propyl)-phenyl]-methanone;
[4-(Cyclohexyl-hydroxy-methyl)-phenyl]-(4-cyclopropyl-[1,4]diazepan-1-yl)-methanone;
(4-Cyclopropyl-[1,4]diazepan-1-yl)-(4-hydroxymethyl-phenyl)-methanone;
(4-Cyclohexanecarbonyl-phenyl)-(4-cyclopropyl-[1,4]diazepan-1-yl)-methanone;
(4-Cyclopropyl-[1,4]diazepan-1-yl)-[4-(hydroxy-phenyl-methyl)-phenyl]-methanone;
(4-Cyclopropyl-[1,4]diazepan-1-yl)-[4-(1-hydroxy-propyl)-phenyl]-methanone;
(4-Cyclopropyl-[1,4]diazepan-1-yl)-[4-(1-hydroxy-2-methyl-propyl)-phenyl]-methanone;
(4-tert-Butyl-phenyl)-(4-cyclobutyl-piperazin-1-yl)-methanone;
(4-Cyclobutyl-piperazin-1-yl)-(4-ethyl-phenyl)-methanone;
(4-Cyclobutyl-piperazin-1-yl)-(4-isopropyl-phenyl)-methanone;
(4-Cyclobutyl-piperazin-1-yl)-(4-cyclohexyl-phenyl)-methanone;
(4-Benzyl-phenyl)-(4-cyclobutyl-piperazin-1-yl)-methanone;
(4-Cyclobutyl-piperazin-1-yl)-(4-propyl-phenyl)-methanone;
(4-Butyl-phenyl)-(4-cyclobutyl-piperazin-1-yl)-methanone;
(4-Cyclobutyl-piperazin-1-yl)-(4-pentyl-phenyl)-methanone;
(4-Cyclobutyl-piperazin-1-yl)-[4-(1-hydroxy-1-methyl-ethyl)-phenyl]-methanone;
(4-Cyclobutyl-piperazin-1-yl)-[4-(1-hydroxy-cyclohexyl)-phenyl]-methanone;
(4-Cyclopropyl-[1,4]diazepan-1-yl)-[4-(1-hydroxy-cyclohexyl)-phenyl]-methanone;
(4-Cyclopropyl-[1,4]diazepan-1-yl)-[4-(1-hydroxy-cyclopentyl)-phenyl]-methanone;
(4-Cyclobutyl-piperazin-1-yl)-[4-(1-hydroxy-cyclopentyl)-phenyl]-methanone;
(4-Cyclopropyl-[1,4]diazepan-1-yl)-[4-(1-hydroxy-cycloheptyl)-phenyl]-methanone; and
[4-(1-Hydroxy-cycloheptyl)-phenyl]-(4-isopropyl-piperazin-1-yl)-methanone;
and pharmaceutically acceptable salts thereof.

14. A compound as defined in claim 1, or a pharmaceutically acceptable salt thereof.

15. A pharmaceutical composition for treating a disease, disorder, or medical condition mediated by histamine H₃ receptor activity, comprising:
(a) an effective amount of a compound of Formula (I): 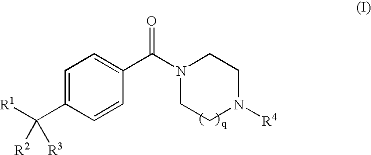
wherein
R¹ is H, C₁₋₄alkyl, monocyclic C₃₋₇cycloalkyl, or phenyl;
R² is H or methyl;
or R¹ and R² taken together form monocyclic C₃₋₇cycloalkyl;
R³ is H, OH, or methyl;
or, when R¹ is not H or phenyl, R² and R³ taken together form a carbonyl;
q is 1 or 2; and
R⁴ is —C₂₋₆alkyl, —C₃₋₆alkenyl, —C₃₋₆alkynyl, monocyclic cycloalkyl, or —C₁₋₂alkyl-(monocyclic cycloalkyl), each unsubstituted or substituted with —OH, —OC₁₋₄alkyl, fluoro, —NH₂, —NH(C₁₋₄alkyl), or —N(C₁₋₄alkyl)₂;
provided that when R¹ is phenyl, and R² and R³ are both H, then q is 1;
or a pharmaceutically acceptable salt, pharmaceutically acceptable prodrug, or pharmaceutically active metabolite thereof; and
(b) a pharmaceutically acceptable excipient.

16. A pharmaceutical composition according to claim 15, further comprising:
an active ingredient selected from the group consisting of H₁ receptor antagonists, H₂ receptor antagonists, H₃ receptor antagonists, serotonin-norepinephrine reuptake inhibitors, selective serotonin reuptake inhibitors, noradrenergic reuptake inhibitors, non-selective serotonin re-uptake inhibitors, acetylcholinesterase inhibitors, and modafinil.

17. A method of treating a subject suffering from or diagnosed with a disease, disorder, or medical condition mediated by histamine H₃ receptor activity, comprising administering to the subject in need of such treatment an effective amount of a compound of Formula (I): 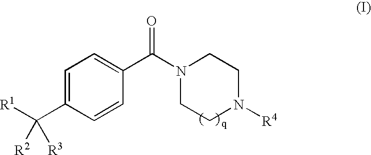
wherein
R¹ is H, C₁₋₄alkyl, monocyclic C₃₋₇cycloalkyl, or phenyl;
R² is H or methyl;
or R¹ and R² taken together form monocyclic C₃₋₇cycloalkyl;
R³ is H, OH, or methyl;
or, when R¹ is not H or phenyl, R² and R³ taken together form a carbonyl;
q is 1 or 2; and
R⁴ is —C₂₋₆alkyl, —C₃₋₆alkenyl, —C₃₋₆alkynyl, monocyclic cycloalkyl, or —C₁₋₂alkyl-(monocyclic cycloalkyl), each unsubstituted or substituted with —OH, —OC₁₋₄alkyl, fluoro, —NH₂, —NH(C₁₋₄alkyl), or —N(C₁₋₄alkyl)₂;
provided that when R¹ is phenyl, and R² and R³ are both H, then q is 1;
or a pharmaceutically acceptable salt, pharmaceutically acceptable prodrug, or pharmaceutically active metabolite thereof.

18. The method according to claim 17, wherein the disease, disorder, or medical condition is selected from the group consisting of: cognitive disorders, sleep disorders, psychiatric disorders, and other disorders.

19. The method according to claim 17, wherein the disease, disorder, or medical condition is selected from the group consisting of: dementia, Alzheimer's disease, cognitive dysfunction, mild cognitive impairment, pre-dementia, attention deficit hyperactivity disorders, attention-deficit disorders, and learning and memory disorders.

20. The method according to claim 17, wherein the disease, disorder, or medical condition is selected from the group consisting of: learning impairment, memory impairment, and memory loss.

21. The method according to claim 17, wherein the disease, disorder, or medical condition is selected from the group consisting of: insomnia, disturbed sleep, narcolepsy with or without associated cataplexy, cataplexy, disorders of sleep/wake homeostasis, idiopathic somnolence, excessive daytime sleepiness, circadian rhythm disorders, fatigue, lethargy, and jet lag.

22. The method according to claim 17, wherein the disease, disorder, or medical condition is selected from the group consisting of: sleep apnea, perimenopausal hormonal shifts, Parkinson's disease, multiple sclerosis, depression, chemotherapy, and shift work schedules.

23. The method according to claim 17, wherein the disease, disorder, or medical condition is selected from the group consisting of: schizophrenia, bipolar disorders, manic disorders, depression, obsessive-compulsive disorder, and post-traumatic stress disorder.

24. The method according to claim 17, wherein the disease, disorder, or medical condition is selected from the group consisting of: motion sickness, vertigo, epilepsy, migraine, neurogenic inflammation, eating disorders, obesity, and substance abuse disorders.

25. The method according to claim 17, wherein the disease, disorder, or medical condition is selected from the group consisting of: depression, disturbed sleep, fatigue, lethargy, cognitive impairment, memory impairment, memory loss, learning impairment, attention-deficit disorders, and eating disorders.

26. A pharmaceutical composition according to claim 15, further comprising topiramate.

27. The method according to claim 17, wherein the disease, disorder, or medical condition is selected from the group consisting of: age-related cognitive decline, REM-behavioral disorder, benign postural vertigo, tinitus, movement disorders, restless leg syndrome, eye-related disorders, macular degeneration, and retinitis pigmentosis.

28. A compound selected from the group consisting of:
(4-Cyclopropyl-piperazin-1-yl)-[4-(1-hydroxy-propyl)-phenyl]-methanone;
(4-Cyclopropyl-piperazin-1-yl)-(4-hydroxymethyl-phenyl)-methanone;
(4-Butyl-piperazin-1-yl)-(4-hydroxymethyl-phenyl)-methanone; and
(4-sec-Butyl-piperazin-1-yl)-(4-hydroxymethyl-phenyl)-methanone;
and pharmaceutically acceptable salts thereof.
